(19) United States
(12) Patent Application Publication
(10) Pub. No.: US 2012/0315674 A1
(43) Pub. Date: Dec. 13, 2012

(54) METHODS OF ENZYMATIC HYDROLYSIS

(75) Inventors: Matthew James REALFF, Atlanta, GA (US); Ronald W. Smith, Ben Lomond, CA (US); Prabuddha Bansal, Atlanta, GA (US); Andreas Sebastian Bommarius, Atlanta, GA (US); Melanie Hall, Atlanta, GA (US); Jay Hyung Lee, Alpharette, GA (US)

(73) Assignee: GEORGIA TECH RESEARCH CORPORATION, Atlanta, GA (US)

(21) Appl. No.: 13/335,535

(22) Filed: Dec. 22, 2011

Related U.S. Application Data
(63) Continuation-in-part of application No. 12/942,906, filed on Nov. 9, 2010.
(60) Provisional application No. 61/259,520, filed on Nov. 9, 2009.

(51) Int. Cl. C12P 19/14; C12P 5/00; C12P 7/64; C12P 7/02

(52) U.S. Cl. 435/99; 435/155; 435/166; 435/134

(57) ABSTRACT

In one embodiment the instant invention generally pertains to a method for producing glucose for fermentation. The method comprises first treating a biomass comprising a lignocellulosic material with a mixture comprising SO2 and steam at reaction conditions sufficient to produce a composition mixture comprising cellulose suitable for enzymatic hydrolysis. Specifically, the temperature, residence time, and SO2 concentration may be selected by calculating a crystallinity index (CrI) of the biomass and using the calculated crystallinity index as an indicator of enzymatic hydrolysis rate. In this manner cellulose may be enzymatically hydrolyzed glucose for aerobic or anaerobic fermentation.

### CROSS-REFERENCE TO RELATED APPLICATIONS

For purposes of United States patent practice, this application claims priority to application Ser. No. 12/942,906 filed on Nov. 9, 2010 and U.S. Provisional Application No. 61/259,520 filed on Nov. 9, 2009 the contents of which are herein incorporated by reference in their entirety to the extent that they are not inconsistent.

### FIELD OF THE INVENTION

The present invention pertains to improved methods for assessing value of a biomass in an enzymatic hydrolysis. The invention also pertains to improved methods of enzymatic hydrolysis which comprise using an initial hydrolysis rate to select or adjust one or more enzymatic reaction conditions.

### BACKGROUND AND SUMMARY OF THE INVENTION

The enzymatic hydrolysis of cellulose to glucose has gained increased interest over the last ten years, and growing demand for economically sustainable biofuels points to an urgent need for reducing costs in their production. Cellulose, a polysaccharide made by many plants, is one of the most abundant organic compounds on Earth and therefore represents a potential goldmine for the biofuel industry. However, current enzymatic degradation of cellulose faces major issues that prevent its wide utilization to produce economically competitive biofuel.

Cellulose is hydrolyzed to glucose via the synergistic action of several enzymes. Endoglucanases (E.C. 3.2.1.4) break down cellulose chains at random positions within the cellulose chains whereas exoglucanases (i.e. cellobiohydrolases, E.C. 3.2.1.91) cleave off cellobiose specifically from the chain ends in a processive manner. Cellobiose is subsequently converted into glucose by β-glucosidase (E.C. 3.2.1.21). The exo-endo synergism is easily understood by the fact that endoglucanases provide more chain ends for cellobiohydrolases to act upon. The hydrolysis of insoluble, solid cellulose is a heterogeneous reaction, which cannot be described by kinetic models based just on Michaelis-Menten kinetics. After an initial phase of adsorption of cellulases on cellulose (fast comparatively to hydrolysis), the enzymes cleave off cellobiose and move along the same chain, hydrolyzing glycosidic bonds until an event that terminates cleavage occurs. As the reaction proceeds to intermediate degrees of conversion, the rate decreases dramatically, and the last part of the cellulose hydrolysis requires an inordinate amount of overall total reaction time. Several factors, both substrate- and enzyme-related, have been suggested to be responsible for this slowdown of rate, but so far no clear picture of what is limiting the reaction has been proposed. The substrate characteristics often implied in the slowdown of rate include surface area, porosity, degree of polymerization, crystallinity, and overall composition (complex substrates such as lignocellulosics vs. pure cellulose). For enzyme-related features, deactivation, inhibition, jamming, clogging, and imperfect processivity are often advanced as causes for slowdown.

One of the most controversial theories concerns the influence of crystallinity and the change of the degree of crystallinity during enzymatic hydrolysis. It is accepted that the initial degree of crystallinity of cellulose plays a major role as rate determinant in the hydrolysis reaction. A completely amorphous sample is hydrolyzed much faster than a partially crystalline cellulose, which has led to the currently widespread assumption that amorphous domains in a partially crystalline cellulose sample are hydrolyzed first, leaving crystalline parts to be hydrolyzed at the end, thus resulting in an increased crystallinity index and explaining the dramatic drop in rate at higher degrees of conversion.

However, studies of this phenomenon differ by the analytical methods (X-ray diffraction vs. solid state ¹³C-NMR), the nature of the substrate used (complex lignocellulosics vs. pure cellulose) and the source of the hydrolytic enzymes (mostly from Trichoderma reesei and other fungal strains). Several reviews have stated that it is still difficult to conclude that crystallinity is a key determinant of the rate of enzymatic hydrolysis. See, for example, (1) Lynd, L. R.; Weimer, P. J.; van Zyl, W. H.; Pretorius, I. S., Microbial cellulose utilization: Fundamentals and biotechnology. Microbiology and Molecular Biology Reviews 2002, 66, (3), 506-+; (2) Zhang, Y. H. P.; Lynd, L. R., Toward an aggregated understanding of enzymatic hydrolysis of cellulose: Noncomplexed cellulase systems. Biotechnology and Bioengineering 2004, 88, (7), 797-824; and (3) Mansfield, S. D.; Mooney, C.; Saddler, J. N., Substrate and enzyme characteristics that limit cellulose hydrolysis. Biotechnology Progress 1999, 15, (5), 804-816. Usually, different types of cellulose with different degrees of crystallinity were employed in these studies (such as cotton, cotton linter, Avicel, filter paper, or bacterial cellulose). Their cellulase-catalyzed degradation gave hydrolysis rates directly related to the initial crystallinity index of the cellulose sample. To correctly relate crystallinity index with hydrolysis rate, it is of prime importance to study samples that have the same basic composition and provenance. More importantly, pure cellulose is superior to complex substrates, as the presence of lignin or hemicellulose may interfere with the cellulase action and reduce accessibility (and therefore hydrolysis rates).

Another important criteria related to hydrolysis rate involves adsorption capacity of cellulases onto cellulose. The rate of hydrolysis has been shown to be proportional to the amount of adsorbed enzymes. Additionally the amount of adsorbed endoglucanases was found to be strictly related to the hydrolyzability of crystalline cellulose. Furthermore, the degree of crystallinity of cellulose influences adsorption at a given protein loading and maximum adsorption constant has been shown to be greatly enhanced with low crystallinity indexes. The same work concluded that the effective binding was the limiting parameter in hydrolysis rate in the case of cellulose with low degrees of crystallinity (despite high adsorption constant).

Amorphous cellulose is a favored modification to investigate cellulase activity. Obtained after treatment with 85% phosphoric acid, the cellulose obtained in this way, PASC (phosphoric acid swollen cellulose), results from complete dissolution of the sample and the treatment was shown to have no impact on the reducing-end concentration of the cellulose sample (i.e. its degree of polymerization). The effect of various phosphoric acid concentrations has only been investigated across a narrow range of acid concentrations or mainly at low concentrations. Recently, Zhang et al. showed that the concentration of phosphoric acid used to generate swollen cellulose dictates the rate of enzymatic hydrolysis by controlling the state of cellulose solubilization (Biomacromolecules 2006, 7, (2), 644-648).

Accordingly, there remains a need for determining the main causes of rate limitations in the enzymatic hydrolysis of, for example, Avicel and other biomass materials, and especially the role of, for example, crystallinity and adsorption on cellulose susceptibility to enzymatic degradation. Both ¹³C-NMR solid-state spectroscopy and X-ray crystallography may be applied to investigate the crystallinity of pure cellulose (Avicel) at different degrees of conversion by cellulases from Trichoderma reesei, the most commonly studied cellulase-producing organism. Cellulose (Avicel) with controlled degrees of crystallinity may be generated with phosphoric acid solutions of precisely calibrated concentration. These cellulose samples from the same source may be employed to investigate and elucidate the relationship between degree of crystallinity, adsorption and enzymatic hydrolysis rates.

Moreover, there remains a need to further enhance the production of biofuels from lignocellulosics. Lignocellulosics are the most abundant natural resource which can be converted to sugars and fermented to biofuel by various microbes. However, biofuel production from lignocellulosics is more complex than producing biofuels from starch. Lignocellulosics are comprised of three main components; cellulose, hemicelluloses, and lignin. These three complex plant polymers are held together tightly by covalent and hydrogen bonds, along with van der Waals forces. This tight packing creates difficulties in the fractionation and hydrolysis of the biomass (Overend & Chomet, 1987). The most severe problem associated with the production of second-generation biofuels is overcoming the recalcitrance of the raw materials. Recalcitrance is a combined effect of several factors: presence of lignin and hemicellulose, reactivity, surface area, pore volume, accessibility, and cellulose crystallinity (R. Sierra, 2008). Much effort has been devoted to developing pretreatment methods to disrupt physical hindrance (such as hemicelluloses and lignin), enhance surface area and accessibility of cellulose to increase yield and attain a commercially viable feedstock (Mosier et al., 2005). Pretreatment methods can be classified into four main categories: physical (e.g. milling and grinding), physicochemical (e.g. steam explosion, hydrothermolysis and wet oxidation), chemical (e.g. alkali, dilute acid and organic solvents), and biological (e.g. white rot fungi pretreatment), with various combinations (Galbe & Zacchi, 2007). Steam explosion is one of the most commonly used pretreatment methods, as it is suitable for a variety of feedstocks and is regarded as one of the most efficient processes (Kumar et al., 2009; Zhu & Pan, 2010). Steam explosion significantly lowers both environmental impact and capital investment (Cara et al., 2006), can be operated in either a batch or continuous manner as a one- or two-step process, and fractionates biomass into its three main constituents (Soderstrom et al., 2004).

Softwood is considered to be the worst-case scenario as a feedstock for the bioconversion process (Pan et al., 2005; Zhao et al., 2008). Its pretreatment at lower-severity conditions optimally liberates easily-hydrolysable hemicelluloses, but generates a solid residue that is not readily amenable to the hydrolysis of cellulase (Lu et al., 2002). Many studies have focused on the optimization of these pretreatment conditions for various feedstocks (Boussaid et al., 1999; Bura et al., 2003; C. Cara, 2008; Carrasco et al., 2011) to maximize sugar recovery. Significantly less work has been performed to elucidate the effects of severity on the constituents of biomass (Boussaid et al., 1999; M. Ibrahim, 2010).

In general terms the instant invention pertains in one embodiment to a method for assessing value of a biomass in an enzymatic hydrolysis comprising first measuring an initial hydrolysis rate of the biomass and then correlating the measured initial hydrolysis rate with an established crystallinity index of standard cellulosic material to project a crystallinity indicative of overall enzymatic hydrolysis susceptibility.

In another embodiment the instant invention generally pertains to a method for producing glucose for fermentation. The method comprises:

(a) treating a biomass with acid and heat under conditions sufficient to produce a composition mixture comprising cellulose suitable for enzymatic hydrolysis;

(b) enzymatically hydrolyzing at least a portion of the cellulose of step (a) under conditions sufficient to form a composition comprising glucose; and

(c) fermenting glucose by, for example, utilizing glucose in an aerobic or anaerobic fermentation process. Typically, one or more reaction conditions of steps (a), (b), or (c) are selected by first measuring an initial hydrolysis rate of said biomass and then selecting one or more appropriate reaction conditions based upon said initial hydrolysis rate.

In yet another embodiment, the invention pertains to a method of using the initial hydrolysis rate to improve the efficiency of an enzymatic hydrolysis. The method comprises:

(a) measuring the initial hydrolysis rate of a biomass to be subjected to hydrolysis and correlating the measured initial hydrolysis rate with an established crystallinity index of standard cellulosic material to project a crystallinity indicative of overall enzymatic hydrolysis susceptibility;

(b) selecting a hydrolysis enzyme and determining the required amount of said enzyme to produce cellulosic sugars with said biomass;

(c) determining the overall hydrolysis rate at said required amount of said enzyme;

(d) selecting or adjusting one or more of the following enzymatic reaction conditions: pre-treatment step, reaction time, reaction temperature, type of enzyme, and amount of enzyme; and

(e) enzymatically hydrolyzing a biomass using one or more of said selected conditions.

In yet another embodiment, the invention pertains to a method for producing glucose for fermentation. The method comprises step (a) of treating a biomass comprising a lignocellulosic material with a mixture comprising SO2 and steam at reaction conditions sufficient to produce a composition mixture comprising cellulose suitable for enzymatic hydrolysis. Next, a portion of the cellulose of step (a) is enzymatically hydrolyzed at least under conditions sufficient to form a composition comprising glucose. Glucose is then utilized in an aerobic or anaerobic fermentation. The temperature, residence time, and SO2 concentration of the treatment step (a) are selected by calculating a crystallinity index (CrI) of said biomass and then using the calculated crystallinity index as an indicator of enzymatic hydrolysis rate.

In yet another embodiment, the invention pertains to a method for producing glucose for fermentation. This method comprises: (a) treating a lignocellulosic material at acidic reaction conditions sufficient to produce a composition mixture comprising cellulose suitable for enzymatic hydrolysis; and (b) enzymatically hydrolyzing at least a portion of the cellulose of step (a) under conditions sufficient to form a composition comprising glucose. The acidic reaction conditions employed in step (a) are selected to reduce the CrIcel (%) of the treated biomass by at least about 6% from that of untreated biomass according to

$${CrI}_{cel} = \frac{CrI}{{F}_{c}}$$

where CrI_{cel} is the CrI of cellulose fraction in treated biomass, F_{c} is the percentage content of cellulose in the sample, and CrI is crystallinity index and wherein the acid insoluble lignin (AIL) content of the treated biomass is less than about 60%.

### BRIEF DESCRIPTION OF THE FIGURES

FIGS. 1(a), 1(b), and 1(c) illustrate effects of phosphoric acid concentration.

FIG. 2 illustrates X-Ray diffraction pattern of two Avicel having differing amounts of crystallinity.

FIG. 3 illustrates effect of crystallinity on initial rate.

FIG. 4 illustrates crystallinity index of Avicel monitored during hydrolysis.

FIG. 5 illustrates SDS-PAGE of extracellular proteins.

FIG. 6 illustrates X-Ray diffraction pattern of untreated Avicel and partially converted cellulose.

FIGS. 7 (a), 7(b), and 7(c)illustrate adsorption, crystallinity index, and initial rates at two cellulase loadings.

FIG. 8a shows the calculated crystallinity vs. enzymatic hydrolysis rate for Avicel.

FIG. 8b shows the calculated crystallinity vs. enzymatic hydrolysis rate for fibrous cellulose.

FIG. 9a shows the histogram of a 42 h hydrolysis for SELP samples.

FIG. 9b shows the SELP samples after normalization to total cellulose content.

FIG. 10 shows the SELP samples adsorption isotherms.

FIG. 11 shows a plot of initial rate as a function of total adsorption capacity.

FIG. 12 shows XRD spectra of PA-treated SELP 2 (PASELP).

FIG. 13 shows as PA concentration increases from 76% to 82%, the CrI of the resulting PASELP decreases linearly (diamond points) and a CrI change of ˜30%-points increases the initial rate fivefold (square points).

FIG. 14 shows a linear relationship between initial rate and CrI.

FIG. 15 shows a correlation plot between initial rates and intensities at different diffraction angles ranging from 10° to 35° in seven differently treated PASELPs.

### DETAILED DESCRIPTION OF THE INVENTION

In general teens the instant invention pertains in one embodiment to a method for assessing value of a biomass in an enzymatic hydrolysis. The method comprises first measuring an initial hydrolysis rate of the biomass that is contemplated for enzymatic hydrolysis. The biomass may be any that is contemplated for enzymatic hydrolysis. That is, the origin and type of the biomass employed is not particularly critical so long as it is capable of undergoing the steps of the instant processes. In one embodiment a particularly preferable feedstock is a lignocellulosic feedstock material such as a plant biomass typically comprised of, for example, cellulose, hemicellulose, poly(aromatics), such as lignin, extractives, ash, and mixtures thereof. Such lignocellulosic materials may often comprise carbohydrate polymers (cellulose and hemicelluloses) which may bond to the lignin. Biomass comes in many different types, which may be grouped into a few main categories: wood or forestry residues, including sawmill and paper mill discards, municipal paper waste, algae, agricultural residues, including corn stover (stalks and straw), and sugarcane bagasse, and dedicated energy crops, which are mostly composed of fast growing tall, woody grasses such as, for example, switchgrass. Any of the aforementioned and others may find use in the instant invention.

The initial hydrolysis rate may be measured by any convenient method so long as it is substantially reliable and/or substantially reproducible. Such methods include, for example, x-ray diffraction, solid state cross-polarization/magic angle spinning nuclear magnetic resonance, infrared spectroscopy, differential scanning calorimetry, or a combination thereof. The use of infrared spectroscopy is described at, for example, Mary L. Nelson & Robert T. O'Connor, Relation of certain infrared bands to cellulose crystallinity and crystal lattice type. Part II. A new infrared ratio for estimation of crystallinity in celluloses I and II, Journal of Applied Polymer Science, 2003, 8, 1325-1341). The use of differential scanning calorimetry (DSC) is described at, for example, Maria Silvia Bertran & Bruce E. Dale, Determination of cellulose accessibility by differential scanning calorimetry, Journal of Applied Polymer Science, 1986, 32, 4241-4253. In one embodiment, the method described in the examples below is employed.

Next, the measured initial hydrolysis rate may be correlated with an established crystallinity index of a standard cellulosic material to project a crystallinity indicative of overall enzymatic hydrolysis susceptibility. The established crystallinity index may be determined by, for example, applying multivariate statistical analysis to X-ray diffraction spectra, solid state cross-polarization/magic angle spinning nuclear magnetic resonance spectra, infrared spectra, differential scanning calorimetry heat capacity temperature plots, or a combination thereof. In one embodiment, multivariate statistical analysis is applied to X-ray diffraction spectra as described in the examples below.

That is, in one specific embodiment to calculate crystallinity indices, normalized X-ray diffraction spectra may be expressed as a linear combination of normalized untreated cellulose (e.g., Avicel or FC) and amorphous cellulose spectra. Principal component analysis (PCA) may then be applied to the spectroscopic data (e.g., separately to Avicel and FC spectra sets) and the principal component scores can then be related to calculated crystallinities. This often reveals the dimensionality of the X-ray spectra data. Cellulose mixtures with varying fractions of untreated and amorphous Avicel as an example may be prepared to validate the predicted crystallinity values. Since initial hydrolysis rates may follow a linear trend with the calculated crystallinity index, principal component regression can likely be used to successfully predict the initial hydrolysis rates from X-ray spectra.

The standard cellulosic material for the established crystallinity index may be selected from any convenient material. In one embodiment a standard cellulosic material is selected from the group consisting of Avicel, fibrous cellulose, bacterial cellulose, cotton, lignocellulosic material, and mixtures thereof. Particularly preferable lignocellulosic materials may include, for example, switchgrass, loblolly pine, bagasse, corn stover, poplar tree, miscanthus, mixtures thereof, etc.

If desired, one may employ further steps which use the measured initial hydrolysis rate. Such steps may include one, two, or more of the following: (a) using the initial hydrolysis rate to determine an amount of hydrolysis enzyme required to produce cellulosic sugars; (b) using the initial hydrolysis rate to determine the overall hydrolysis rate with a particular quantity of hydrolysis enzymes; and (c) using the initial hydrolysis rate to select one or more reaction conditions selected from the group consisting of pre-treatment, reaction time, reaction temperature, type of enzyme, and amount of enzyme. In this manner, one may enzymatically hydrolyze a biomass of virtually any type in a more efficient manner using one or more the selected or adjusted conditions.

In another embodiment, the instant invention pertains to a method for producing glucose. The glucose may be fermented by, for example, utilizing glucose in an aerobic or anaerobic fermentation process. The method comprises first (a) treating a biomass with acid and heat under conditions sufficient to produce a composition mixture comprising cellulose suitable for enzymatic hydrolysis. The biomass may be selected from, for example, any of those described above. The type of acid, amount of heat and other treatment conditions are not particularly critical so long as the result is a composition mixture comprising cellulose suitable for enzymatic hydrolysis. A number of such suitable conditions are described in the literature references and in the examples below.

Next, in step (b) at least a portion of the cellulose of step (a) is enzymatically hydrolyzed under conditions sufficient to form a composition comprising glucose. The conditions of enzymatic hydrolysis are not particularly critical so long as a composition comprising glucose results and a wide variety of suitable conditions are described in the literature references and in the examples below. The glucose may then be utilized in an aerobic or anaerobic fermentation process using suitable conditions to form a desired fermentation product or products. Such a product or products may be selected from the group consisting of alcohols, fatty alcohols, hydrocarbons, fatty acids, tryglycerides, terpenes, and combinations thereof.

As described above, there is a wide variety of conditions available for steps (a), (b), and (c) of the previously described method. One of the advantages of the present method is that the conditions may be selected or determined in order to make any one or more of steps (a), (b), or (c) more efficient. That is, one or more reaction conditions of steps (a), (b), or (c) may be selected or determined by first measuring an initial hydrolysis rate of a biomass and then selecting one or more appropriate reaction conditions based upon said initial hydrolysis rate. In one embodiment, one or more appropriate conditions may be determined by correlating the measured initial hydrolysis rate with an established crystallinity index of standard cellulosic material to project a crystallinity indicative of overall enzymatic hydrolysis susceptibility. These one or more appropriate conditions may be selected from, for example, the group consisting of type of enzyme, amount of enzyme, reaction time, and reaction temperature.

As described above and in the examples below, an initial hydrolysis rate may be measured by, for example, x-ray diffraction, solid state cross-polarization/magic angle spinning nuclear magnetic resonance, infrared spectroscopy, differential scanning calorimetry, or a combination thereof. And similarly the established crystallinity index may be determined by, for example, applying multivariate statistical analysis to X-ray diffraction spectra, solid state cross-polarization/magic angle spinning nuclear magnetic resonance spectra, infrared spectra, differential scanning calorimetry heat capacity temperature plots, or a combination thereof.

In another embodiment, the invention pertains to a method of using the initial hydrolysis rate to improve the efficiency of an enzymatic hydrolysis. The method comprises first measuring the initial hydrolysis rate of a biomass to be subjected to hydrolysis and correlating the measured initial hydrolysis rate with an established crystallinity index of standard cellulosic material to project a crystallinity indicative of overall enzymatic hydrolysis susceptibility. The selection of biomass, as well as, the measurement and correlation of the hydrolysis rate are as described above.

Next, the hydrolysis enzyme and amount useful to produce cellulosic sugars with the biomass is determined along with the overall hydrolysis rate for the amount of enzyme. Enzymatic reaction conditions may then be selected or adjusted based upon, for example, a measured initial hydrolysis rate and/or correlation with crystallinity index. Such conditions include, for example pre-treatment step, reaction time, reaction temperature, type and amount of enzyme, etc. The biomass may then be enzymatically hydrolyzed using one or more of said selected or adjusted conditions. In many cases, the hydrolysis may be more efficient or optimal using the aforementioned methods.

In another embodiment, the instant invention may be beneficial in, for example, selecting a biomass having a suitable crystallinity for a contemplated hydrolysis for further study or implementation. In this manner, one may efficiently assess the value of a given material for enzymatic hydrolysis in a small amount of time. In alternative embodiments, the method may be accomplished in less than 1 day, or less than 12 hours, or less than 1 hour, or less than 10 minutes, or less than 5 minutes, or in some cases in less than 2 minutes. In some embodiments such a method may be accomplished without resort to extensive x-ray crystallography, solid-state NMR, or enzymatic hydrolysis which methods may take much longer and/or be more expensive and/or require more specialized equipment. The instant method may be a quick and useful predictor of a given biomass's ease of hydrolyzability, overall reaction time, and/or overall amount enzyme that may be required. This advantageously allows the practitioner to quickly screen many potential biomass sources to find ones that may be more useful for hydrolysis by exhibiting, for example, a suitable crystallinity.

In another embodiment the instant invention generally pertains to a method for improved enzymatic hydrolysis of biomass. This method comprises first measuring an initial hydrolysis rate of a biomass and then using the initial hydrolysis rate to select or adjust one or more of the following enzymatic reaction conditions: type of biomass, pre-treatment step (if employed), reaction time, reaction temperature, type of enzyme, and amount of enzyme. Next, a biomass is enzymatically hydrolyzed using one or more of said selected or adjusted conditions. In some embodiments, the method may be accomplished without resort to extensive x-ray crystallography, solid-state NMR, or enzymatic hydrolysis which methods may take much longer and/or be more expensive and/or require more specialized equipment.

### Enzymatic Hydrolysis of Lignocellulosic Materials

In one embodiment the instant invention may be employed to produce glucose for fermentation from a biomass comprising a lignocellulosic material. The specific lignocellulosic material is not particularly critical so long as it is capable of producing a composition mixture comprising cellulose suitable for enzymatic hydrolysis under suitable reaction conditions. Suitable lignocellulosic materials may often be selected from the group consisting of switchgrass, loblolly pine, bagasse, corn stover, poplar tree, miscanthus, spruce, eucalyptus, and mixtures thereof.

The initial treatment conditions to produce a composition mixture comprising cellulose suitable for enzymatic hydrolysis from the biomass comprising lignocellulosic material may vary widely depending upon many factors. Among these include the type of lignocellulosic material or mixture, the amount and type of hydrolysis enzyme to be employed, and the desired fractionation of the biomass. One particularly useful treatment is employing heat and acidic conditions to the lignocellulosic material.

The amount of heat, as well as type and concentration of acid will vary depending upon, for example, the biomass comprising lignocellulosic material. Advantageously, one may select more advantageous acidic conditions by calculating a crystallinity index (CrI) of said biomass and then using the calculated crystallinity index as an indicator of enzymatic hydrolysis rate. Typically, one would select acidic conditions which are severe enough to, for example, lower the crystallinity index from that of untreated lignocellulosic material. However, one would not select acidic conditions so severe as to result in, for example, excessively high proportions of acid insoluble lignin in the treated material as this can often result in decreased subsequent hydrolysis rate. Of course, the actual quantitative lignin amounts vary since the amount of lignin varies by material, i.e., the woody trees generally have a much higher lignin content than, for example, a switchgrass.

The severity of acidic treatment conditions is determined by a number of factors. In general, the severity increases with an increased amount of residence time (time of lignocellulosic material exposure to the conditions). Similarly, the severity increases depending upon pH (the lower the pH the more severe acidic conditions). And generally the higher the treatment temperature and pressure, the more severe the treatment.

The acidic conditions may be achieved by any convenient method or combination of methods and may be defined by, for example, pH, residence time, and temperature. Suitable methods for achieving acidic conditions include employing heat and/or aqueous acid like phosphoric acid, using steam and SO2 which is sometimes referred to as steam explosion, and combinations thereof.

No matter the method or methods employed it is often useful to select one which reduces the CrIcel (%) of the treated biomass from that of untreated biomass according to

$${CrI}_{cel} = \frac{CrI}{{F}_{c}}$$

where CrI_{cel} is the CrI of cellulose fraction in treated biomass, F_{c} is the percentage content of cellulose in the sample, and CrI is crystallinity index. The amount that CrI_{cel} is reduced will vary. Often it is advantageous to select conditions such that the CrIcel (%) of the treated biomass is reduced by at least about 6%, or at least about 12%, or at least about 15%, or at least about 18% or even at least about 20% from that of untreated biomass. In example 5 below, SELP2 conditions resulted in the treated biomass being reduced by 6.5% (58.2-51.7) from that of untreated biomass while SELP3 conditions resulted in the treated biomass being reduced by 19.5% (58.2-38.7) from that of untreated biomass.

The acidic conditions advantageously selected for lignocellulosic materials may be defined in terms of severity index (R) wherein R is calculated according to the following Equation 1:

$$R = {log}_{10} ( t {e}^{\frac{T - 100}{14.75}} ) - pH \qquad ( 1 )$$

where t is residence time (min) and T is temperature (° C.). Often for lignocellulosic materials and particularly woody cellulosic materials like loblolly pine it may be advantageous to select conditions which yield a severity index of at least about 2.5, or at least about 2.8, or at least about 3.1. On the other hand, it is often useful for lignocellulosic materials and particularly woody cellulosic materials like loblolly pine to select conditions which yield a severity index of less than about 4.0, or less than about 3.8, or less than about 3.6. For example, when employing steam explosion one may select a combination of temperature, residence time, and SO2 concentration to yield the desired severity index. As described in example 5, for SLEP1 a 2.9% SO2 coupled with a temperature of 419F with a 5 minute residence time results in an R=3.1 severity.

As described above, the severity of the acidic treatment may cause an increase in insoluble lignin (AIL) content. If the increase it too large then it may be disadvantageous because the hydrolysis is adversely affected. When using steam explosion on lignocellulosic materials it has been found to be advantageous to select conditions such as a temperature, residence time, and SO2 concentration to yield a severity index (R) of at least about 3.2. Moreover, it is also advantageous in many cases to select and/or modify the conditions such that an acid insoluble lignin (AIL) content of the treated biomass is less than about 60%.

In the same vein, when the lignocellulosic material is selected from the group consisting of loblolly pine, poplar tree, spruce, eucalyptus, and mixtures thereof it is often advantageous to select conditions such as temperature, residence time, pH (e.g., SO2 concentration) in order to maintain the acid insoluble lignin (AIL) content of the treated biomass less than about 40% or between about 25 and about 70%. In this manner, efficient enzymatic hydrolysis to obtain glucose is often obtained.

The glucose obtained may be fermented in any convenient manner such are aerobic, anaerobic, or some combination thereof. The products obtained include, for example, alcohols, fatty alcohols, hydrocarbons, fatty acids, tryglycerides, terpenes, and combinations thereof.

### LEGENDS FOR FIGURES IN EXAMPLES

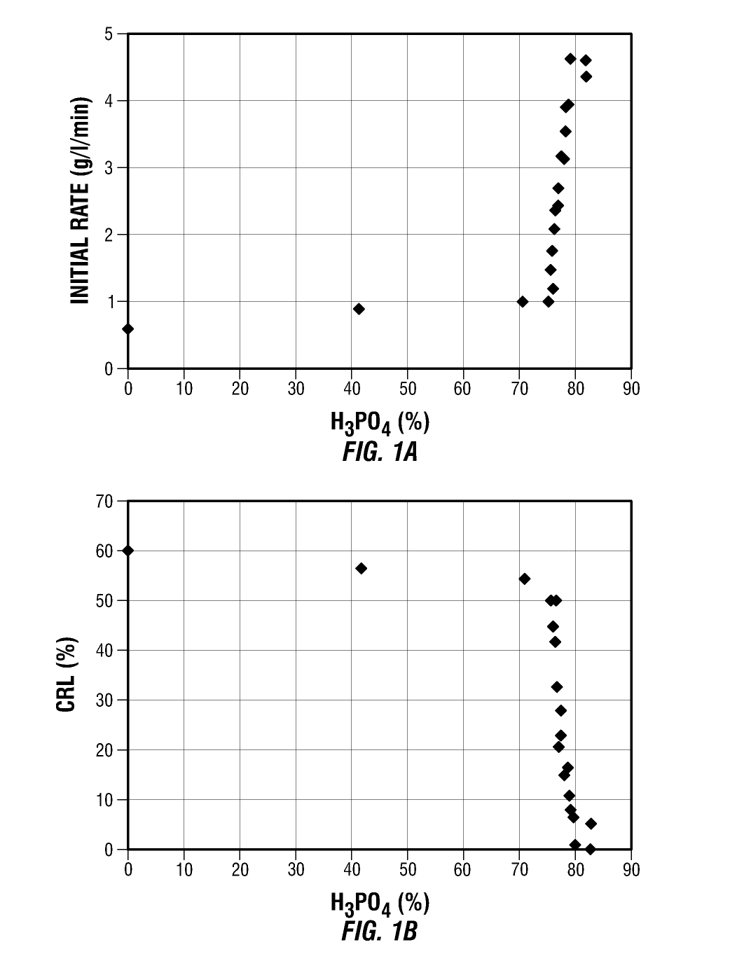

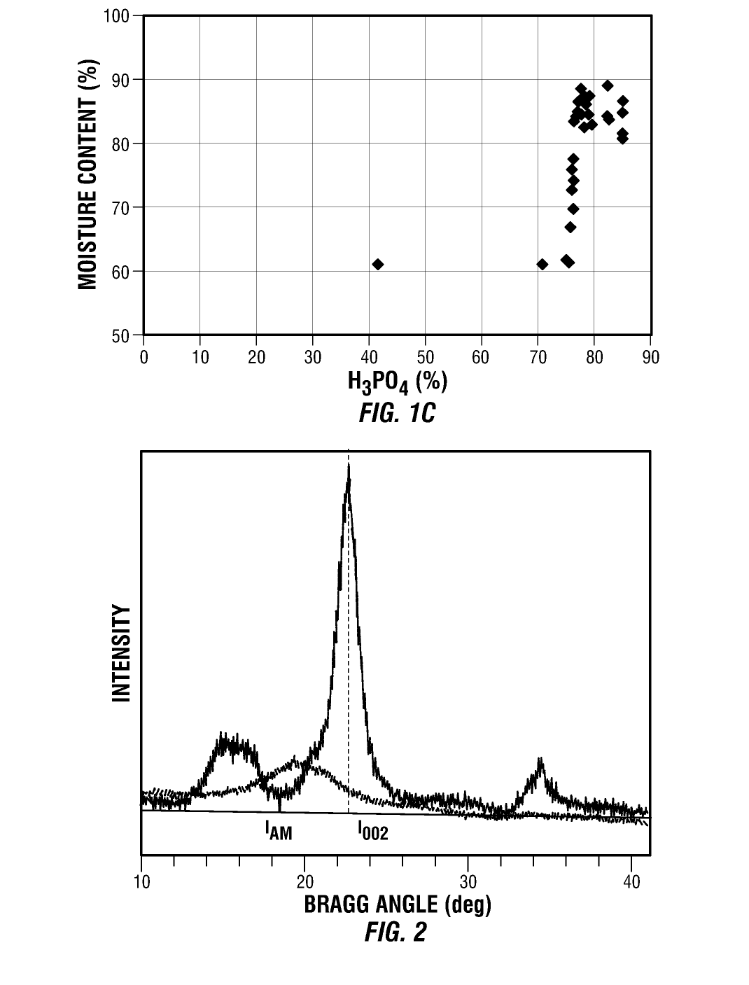

FIG. 1. a. Effect of phosphoric acid concentration on: a. initial rate of Avicel enzymatic hydrolysis (glucose produced in the first 2 min of the reaction with cellulases); b. crystallinity index CrI obtained from X-ray diffraction data; c. moisture content of cellulose samples after treatment with phosphoric acid (measurement performed after tightly controlled filtration and subsequent drying at 60° C.).

FIG. 2. X-Ray diffraction pattern of microcrystalline cellulose Avicel (red) and amorphous Avicel (blue) generated with 85% phosphoric acid (reflection around 20° is attributed to amorphous parts and gives CrI=0% based on peak intensity method⁷¹). X-axis: Bragg angle (2θ). I₀₀₂ represents the maximum intensity at 2θ=22.5°, Iₐₘ shows the minimum intensity at 2θ=18° used to calculate crystallinity in the peak height method, and the straight line represents the background (see corresponding experimental section).

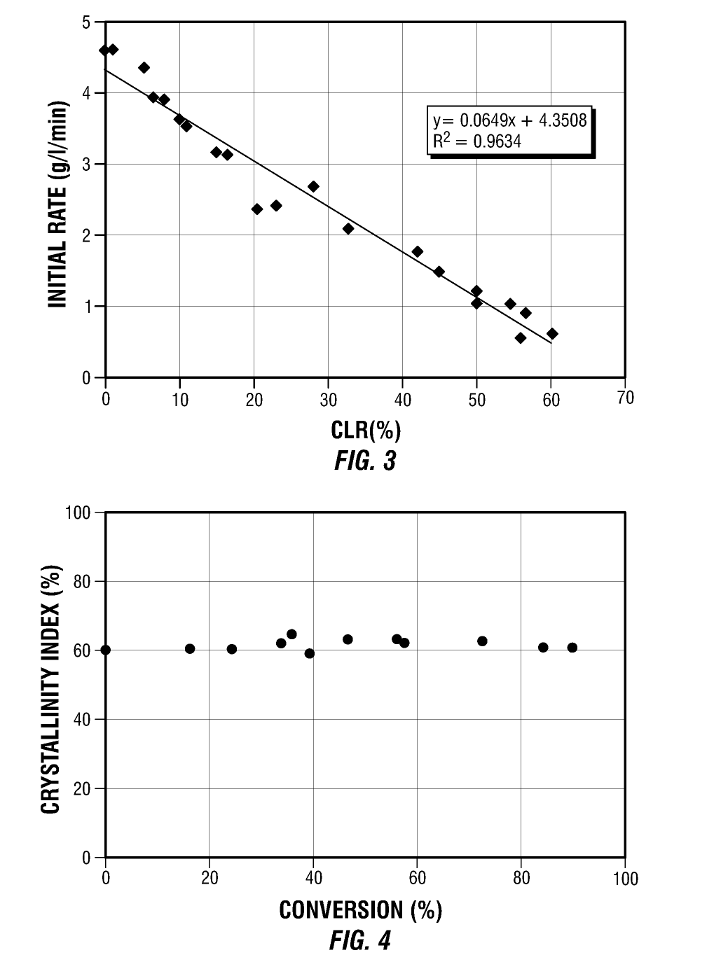

FIG. 3. Effect of crystallinity on the initial rate in Avicel enzymatic hydrolysis (glucose produced in the first 2 min of the reaction with cellulases).

FIG. 4. Crystallinity index of Avicel monitored during hydrolysis with cellulases via CP/MAS ¹³C-NMR.

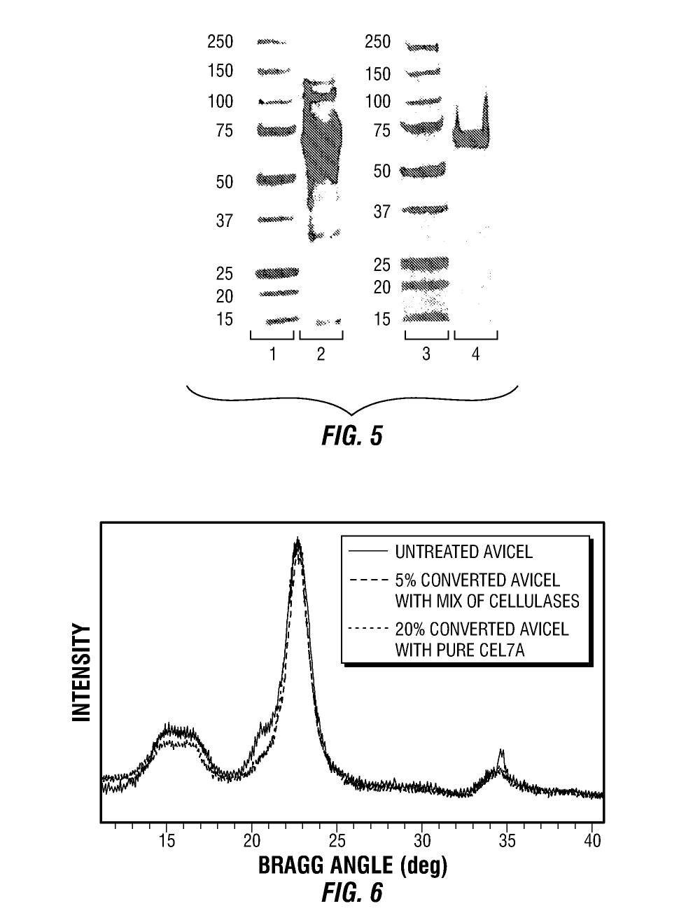

FIG. 5. SDS-PAGE of extracellular proteins produced by Trichoderma reesei, and purified Cel7A. Lanes 1 and 3: ladder, lane 2: proteins in the liquid medium containing overexpressed Cel7A (after cells were filtered off) and lane 4: Cel7A purified by anion-exchange chromatography (67 kDa)

FIG. 6. X-ray diffraction patterns of untreated Avicel and partially converted cellulose between 10 and 40° (2θ). X-axis: Bragg angle (2θ). The reflection of face (021) of the crystal (centered around 21)° is visible only for untreated Avicel.

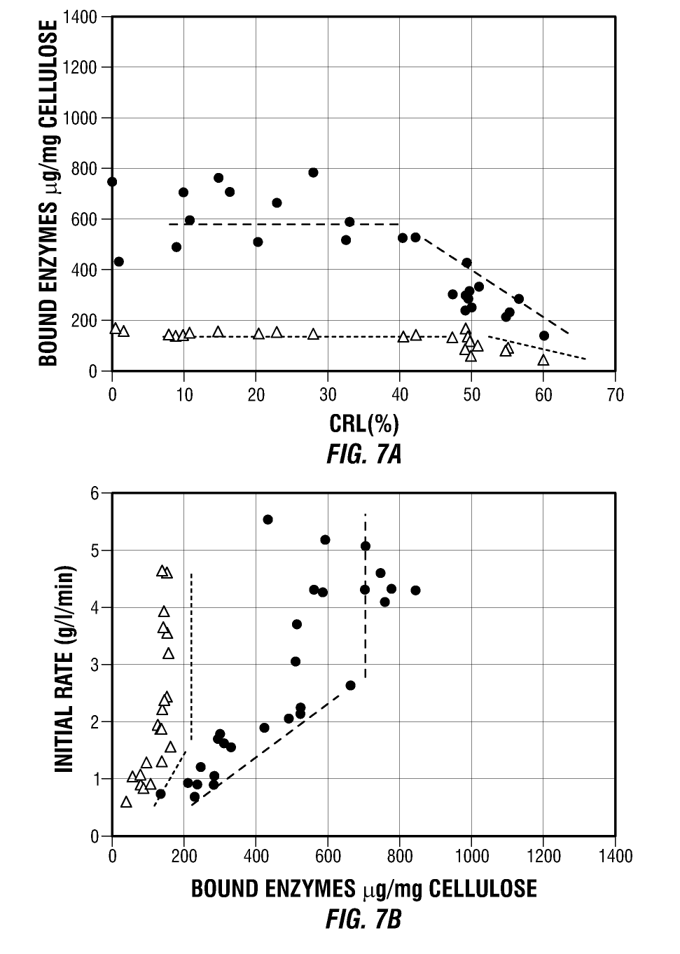

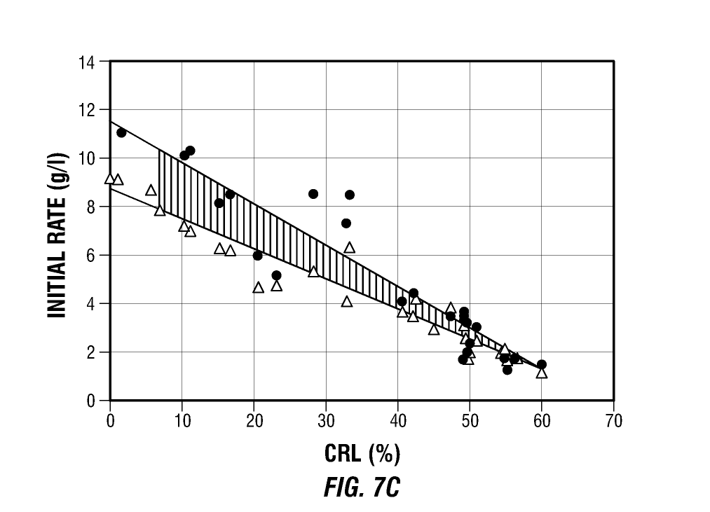

FIG. 7. Adsorption, crystallinity index and initial rates at two cellulases loading: 175 μg/mg cellulose; 1230 μg/mg cellulose. Initial rates correspond to the amount of glucose produced over a 2 min reaction (20 mg cellulose/ml, corresponding amount of cellulases and excess of β-glucosidase, 50° C.). Adsorption studies were conducted at 4° C. over 30 min incubation time. a. adsorption vs crystallinity index; b. initial rate vs adsorption; c. initial rate vs crystallinity index, where grey shaded area represents the importance and role of adsorption on enzymatic rate.

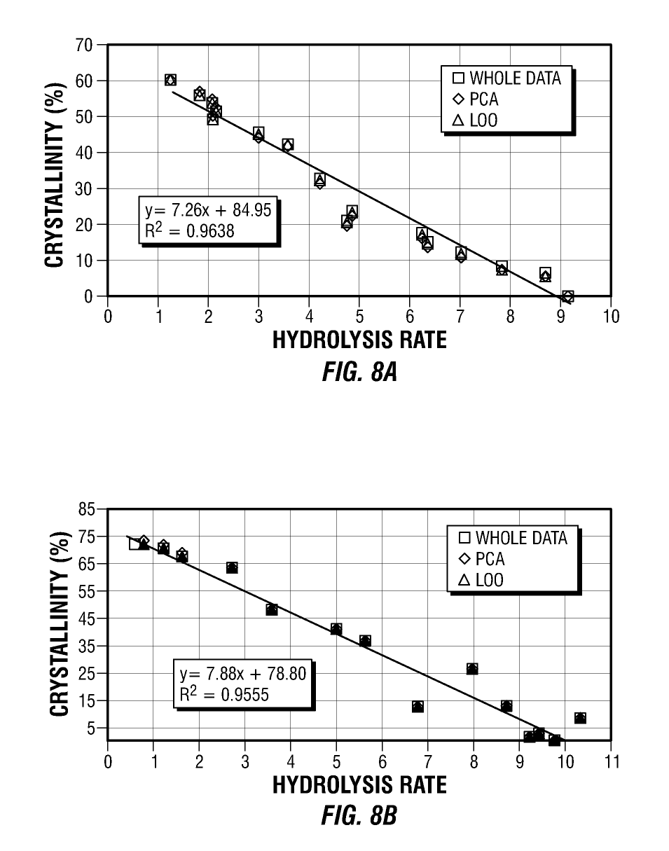

FIG. 8. Calculated crystallinity vs. enzymatic hydrolysis rate. The hydrolysis rates correspond to the amount of glucose produced in the first 2 min of the reaction with cellulases. The linear equations shown are the fits between degrees of crystallinity calculated with whole spectra and hydrolysis rates.

### Example 1

### Avicel

### Material and Chemicals

All chemicals and reagents were purchased from Sigma unless otherwise stated. Avicel PH-101, cellulases from Trichoderma reesei (159 FPU/ml) and β-glucosidase (from almonds, 5.2 U/mg) were from Sigma, phosphoric acid (85%) was from EMD. Trichoderma reesei QM9414 strain was obtained from ATCC (#26921). BCA protein assay kit was obtained from Thermo Fischer Scientific.

### Phosphoric Acid Pretreatment

1 g of slightly moistened Avicel was added to 30 ml of an ice-cold aqueous phosphoric acid solution (concentration ranging from 42% up to 85% wt) and allowed to react over 40 min with occasional stirring. After addition of 20 ml of ice-cold acetone and subsequent stirring, the resulting slurry was filtered over a fritted filtered-funnel and washed three times with 20 ml ice-cold acetone, and 4 times with 100 ml water. The resulting cellulose was used as such in enzymatic hydrolysis experiments, after moisture content was estimated upon oven-drying at 60° C. overnight. Samples were freeze-dried prior to X-ray diffraction measurements.

### Enzymatic Hydrolysis of Cellulose

A suspension of Avicel (20 g/p) in sodium acetate buffer (1 ml, 50 mM, pH 5) was hydrated during 1 h under stirring at 50° C. β-Glucosidase (15 kU/1) and cellulases (24 ml/l, 3.4 g/l total protein) were added and the mixture was stirred at 50° C. At desired time points, samples were centrifuged, and glucose content in the supernatant was measured via the dinitrosalicylic acid (DNS) assay. For crystallinity measurements at various conversion levels, reactions were run on a 15 ml scale and after centrifugation and washing with buffer and water, recovered cellulose was either freeze-dried, oven-dried (60° C.) or air-dried. When Cel7A was used as single cellulase component, 92 μg of purified enzyme per mg of Avicel were added to the reaction mixture.

### Determination of Glucose Content

Glucose released from cellulose was measured using the DNS assay, as published before²⁸. The calibration curve was generated with pure glucose standards. DNS assay was compared to HPLC analysis and was found to yield identical conversion results.

### Determination of the Degree of Crystallinity of Cellulose

### X-Ray Diffraction

X-ray diffraction patterns of cellulose samples obtained after freeze-drying were recorded with an X′Pert PRO X-ray diffractometer at room temperature from 10-60°, using Cu/Kα₁ irradiation (1.54 Å) at 45 kV and 40 mA. Scan speed was 0.021425°/sec with a step size of 0.0167°. CrI was calculated using peak intensity method⁷¹:

$$CrI = \frac{{I}_{002} - {I}_{am}}{{I}_{002}} \times 100$$

where I₀₀₂ is the intensity of the peak at 2θ=22.5°, and Iₐₘ is the minimum in intensity corresponding to amorphous content at 2θ=18°. Freeze-drying showed no impact on the crystallinity of untreated Avicel.

### Solid State ¹³C-NMR

The solid-state cross polarization/magic angle spinning (CP/MAS) ¹³C-NMR experiments were performed on a Bruker Avance/DSX-400 spectrometer operating at frequencies of 100.55 MHz for ¹³C. All the experiments were carried out at ambient temperature using a Bruker 4-mm MAS probe. The samples (˜35% moisture content) were packed in 4 mm zirconium dioxide rotors and spun at 10 kHz. Acquisition was carried out with a CP pulse sequence using 5 μs pulse and 2.0 ms contact pulse. CrI was calculated according to literature²⁸

$$CrI = \frac{{A}_{86 - 92 ppm}}{{A}_{79 - 86 pp m} + {A}_{86 - 92 pp m}} \times 100$$

where A₈₆₋₉₂ₚₚₘ and A₇₉₋₈₆ₚₚₘ are the areas of the crystalline, resp. amorphous C4 carbons signal of cellulose.

Oven-drying (60° C.) showed no impact on the crystallinity of untreated Avicel.

### Multivariate Statistical Analysis of X-Ray Data

The crystallinity index of cellulose samples was calculated by quantifying the contribution of amorphous cellulose (PASC) and Avicel to its (normalized) X-ray diffraction spectra. A full discussion may be found in Bansal, P.; Hall, M.; Realff, M. J.; Lee, J. H.; Bommarius, A. B., Multivariate statistical analysis of X-ray data from cellulose: A new method to determine degree of crystallinity and predict hydrolysis rates. 101 Bioresource Technol. 4461-4471 (2010) and/or Hall et al., “Cellulose Crystallinity—a key predictor of the enzymatic hydrolysis rate”, FEBS Journal 277 (2010) 1571-1582, each of which is incorporated by reference herein for purposes of U.S. patent practice.

Iⱼ(2θ)=fⱼI_{P}(2θ)+(1−fⱼ)I_{C}(2θ)+ε

Where Iⱼ (2θ) is the intensity of the j^{th} sample at diffraction angle 2θ, Iₚ (2θ) is the intensity of PASC at diffraction angle 2θ, I_{C} (2θ) is the intensity of untreated Avicel at diffraction angle 2θ, fⱼ is the contribution of PASC to the spectrum, ε is the random error.
\hat{f}ⱼ, the least square estimate of fⱼ, was used to estimate the crystallinity by multiplying the contribution of Avicel (1−\hat{f}ⱼ) by its crystallinity (calculated by CP/MAS ¹³C-NMR to be 60%):

Criⱼ=(1−\hat{f}ⱼ)*Cri_{C}

where Criⱼ is the crystallinity (in percentage) of the j^{th} sample of Avicel, Cri_{C} is the crystallinity of Avicel (60%).

Crystallinity was also calculated after principal component analysis (PCA) of the X-ray data set of all the samples. It was found that only one principal component was sufficient to capture the variation in the data sets. The reconstructed spectra (from one principal component) were then used in the equations above to calculate the crystallinity, and the values were found to be very close to the ones calculated by using the whole spectra.

### Cel7A Purification

Trichoderma reesei QM9414 was grown on potato dextrose agar plate under light illumination. Spores were harvested and used to inoculate the liquid medium (minimal medium: (NH₄)₂SO₄ 5 g/l, CaCl₂ 0.6 g/l, MgSO₄ 0.6 g/l, KH₂PO₄ 15 g/l, MnSO₄.H₂O 1.5 mg/l, FeSO₄.7H₂O 5 mg/l, COCl₂ 2 mg/l, ZnSO₄ 1.5 mg/l) supplemented with glucose (2%). After 3 days at 28° C. and 150 rpm, the fungus was grown on lactose (2%) in minimal medium for up to 12 days at 28° C. and 150 rpm. After filtration over glass-microfiber filter (GF/A 1.6 μm), the filtrate was diafiltered by repeated concentration and dilution with sodium acetate buffer (50 mM, pH 5.5) using a polyethersulfone membrane (MWCO 10 kDa). The concentrate was purified by means of anion-exchange chromatography using a Q-Sepharose Fast Flow with a 10 mM to 500 mM sodium acetate gradient (pH 5.5). Cel7A was eluted in the last peak, and purity was confirmed by SDS-PAGE. Enzyme concentrations were estimated by the Bradford assay, using bovine serum albumin as standard.

### Adsorption Study

Cellulose samples (20 mg/ml) in NaOAc buffer (50 mM, pH 5) were incubated at 50° C. for 1 h under agitation (900 rpm), then cooled down to 4° C. Cellulases were added in various amounts and the mixture was further agitated for 30 min. After centrifugation, the supernatant was collected and protein content analysis was performed using the BCA protein assay (as described in Pierce® BCA Protein Assay Kit.

### Results and Discussion

### Cellulose Crystallinity and Cellulase Hydrolysis Rate

Various types of (ligno)cellulosic substrate are employed in current enzymatic hydrolysis studies and thus are a source of discrepancies in results and potential confusion to the challenging problem of understanding cellulase mode of action³¹. The presence of hemicellulose, and especially lignin, a strong adsorbent on cellulose, in lignocellulosics often interferes with the enzymatic activity of cellulases on cellulose^{14, 29, 38}. To avoid such interference, we used Avicel, a commonly used, commercially and reproducibly obtainable pure cellulose substrate with well-characterized structure and an average degree of crystallinity of 60% (measured via solid state ¹³C-NMR).

### Phosphoric Acid Pretreatment

First, to validate the efficiency of the phosphoric acid pretreatment, acid pretreated samples were hydrolyzed with cellulases (and an excess of β-glucosidase to remove product inhibition and fully convert cellobiose to glucose) and initial hydrolysis rate was calculated as production of glucose after 2 min reaction time. As expected, the more concentrated the phosphoric acid solution, the higher the sugar production (FIG. 1a), so that the pretreatment procedure was regarded as efficient. Samples treated with pure phosphoric acid solution (max. 85%) gave amorphous cellulose as proven by X-ray diffraction analysis⁷⁴ (FIG. 2, blue diffraction pattern). Besides, a high amount of glucose (4.75 g/l/min) was produced from the cellulose sample pretreated with the most concentrated acid solution (85%), and all Avicel was converted in less than 2.5 h, compared to over 96 h necessary for untreated Avicel (data not shown).

Phosphoric acid pretreatment has been used to create cellulose samples of various surface areas and this parameter was found to be related to enzymatic rate⁴⁹. A recent study using phosphoric acid to increase cellulose accessibility in lignocellulosics suggested the presence of a critical point in phosphoric acid concentration below which enzymatic hydrolysis was slow, and above which cellulose was easily dissolved⁵⁶. Our own results (FIGS. 1 a-c) confirm that there is a steep change in reactivity (i.e. glucose production) from 1 to 4.75 g/l/min (FIG. 1a), not as a step change, but a steep continuum in a narrow range of phosphoric acid content between 75 and 80%. No further increase was observed beyond 80% (with a maximum phosphoric acid concentration possible of 85%, as commercially available), close to the 81% obtained by Moxley et al. for maximum glucan digestibility⁵⁶. Below 75%, the glucose production rate tends to level off to reach a minimum obtained with untreated Avicel (0.6 g/l/min glucose at 0% phosphoric acid). Thus, instead of aiming at the highest possible acid concentration for pretreating cellulose, one should decide first which hydrolysis rate is economically viable for a process and then select accordingly the corresponding pretreatment conditions to reach the desired rate.

There are several ways to measure cellulose crystallinity index (CrI); one of the most commonly employed techniques is X-ray diffraction where the peak height is used to calculate CrI⁷¹ (as depicted in FIG. 2). However, the major drawback of that analytical method comes from the formula itself (see corresponding experimental section), as it implies that amorphous cellulose gives a main reflection at 2θ=18°, which upon our analysis is definitely not the case (it is rather shifted to higher angle, ca. 19.5°). Also, the absolute values thus obtained are extremely high (>90% for Avicel), which does not seem to represent well the structure of Avicel and deviate largely from NMR analysis (60% for Avicel). In addition, the literature gives a full range of numbers for the same sample Avicel using X-ray diffraction (ranging from 62 to 87.6% using the peak height method⁵⁷⁻⁵⁹, and from 39 to 75.3% using various others^{57, 60, 73}). Under our conditions, no satisfactory resolution of the C4 carbons signals in NMR analysis could be obtained below a certain degree of crystallinity (and within reasonable acquisition time), so that X-ray diffraction was used as alternative to map the full crystallinity spectrum. Since there may be some issues with the peak intensity method to calculate degree of crystallinity from X-ray diffraction (usually values are much higher than those obtained with NMR⁶¹ or with integrated area method, overall intensities may vary from one measurement to the other⁷², plus the need to define the background baseline seems arbitrary and subject to user-based deviations; it remains a relative technique⁶²), we have developed a new method to obtain consistent crystallinity index values using multivariate statistical analysis applied to X-ray diffraction spectra⁷⁴ and the values presented here were obtained from that analytical method.

FIG. 1b shows that CrI closely tracks the breakthrough behavior of reactivity (FIG. 1a) at the same amount of phosphoric acid used to pretreat the cellulose sample: the degree of crystallinity remains fairly unchanged at around 55-60% over a wide range of phosphoric acid concentrations but decreases linearly to nearly 0% within the same concentration range of 75 to 80% phosphoric acid mentioned above. Thus, the phosphoric acid effect is clearly evident: not only is it related to dissolution capacity⁵⁶, it also disrupts the crystalline structure of cellulose and can turn partially crystalline cellulose amorphous. Avicel, a microcrystalline type of cellulose, has a mixed composition (amorphous and crystalline) and our results suggest that there is a gradual transition between the two states. The more concentrated the acid solution, the more crystalline regions are turned amorphous. The capacity of cellulose samples to retain water relative to the proportion of amorphous parts has been postulated^{62, 63}, and was verified with the acid-treated samples. FIG. 1c shows the tight relationship between moisture content and acid concentration, supporting the conclusion on structural changes derived from crystallinity measurement, and which happen in the 75-80% acid concentration range. Upon treatment at higher acid concentrations, cellulose samples have a higher capacity to retain water, owing to the higher number of hydroxyl groups available to bind to (and adsorb) water molecules, as they are not interacting with each other anymore. A cellulose sample with 85% moisture content can theoretically accommodate 49 water molecules per glucose unit, whereas a 60% moisture content reduces this ratio down to 13 (based on the observation that 1 g Avicel yields 1.15 g of glucose at 100% conversion).

The correlation between crystallinity index and initial hydrolysis rate (FIG. 3) shows a continuous and ever-accelerating decrease in rate as crystallinity increases. The higher the degree of crystallinity, the less cellulose samples are amenable to enzymatic hydrolysis, the less reactive they are, and the less accessible. The latter is supported by the data obtained from moisture content measurement (FIG. 1c). Most aqueous reagents can only penetrate the amorphous parts of cellulose; therefore, these domains are also called the accessible regions of cellulose, and crystallinity and accessibility are closely related⁶². It seems likely that crystallinity and accessibility are related, but looking at moisture content (capacity to retain water) cannot be translated in term of enzyme accessibility, as water molecules are three orders of magnitude smaller than cellulases⁶⁴. A highly crystalline cellulose sample has a tight structure with cellulose chains closely bound to each other, leaving too little space for enzymes to start the hydrolysis process anywhere within the cellulose crystal.

Overall, the hydrolysis rate vs. phosphoric acid concentration profile resembles a very steep and sharp sigmoid curve (FIG. 1a), so we inspected more closely the concentration range corresponding to the sigmoid region. In their review, Zhang et al. stressed that crystallinity index of cellulose was not strongly associated with hydrolysis rates³¹. Our results, on the contrary, show a very close and linear relationship between crystallinity index and initial hydrolysis rate for samples of same origin obtained after pretreatment with phosphoric acid (R²=0.96, FIG. 3), showing that crystallinity is a good predictor of hydrolysis rate. More precisely, in the region between 75 and 80% phosphoric acid concentration, hydrolysis rate, crystallinity, and phosphoric acid concentration are mutually dependent parameters describing and resulting from the structural changes happening upon acid pretreatment of cellulose and related to one another by linear relationships.

We showed that phosphoric acid enables a tight control of the overall structure of cellulose in Avicel sample, which might be of importance in kinetics studies where estimation of cellulose intrinsic parameters is needed.

### Cellulose Enzymatic Hydrolysis

There have been numerous controversial studies on the change of cellulose crystallinity upon enzymatic hydrolysis. Both trends (increased degree of crystallinity over conversion, no change over conversion) were observed at different levels of intensity^{14, 36, 65-67}. As mentioned above, both the different types of substrate and analytical methods employed contributed to the absence of clear understanding of the mechanism of cellulases on partially crystalline cellulose. Besides, in-situ measurements of cellulose structure under reacting conditions (in aqueous buffers) are difficult as all current methods require prior isolation of cellulose and drying to some extent²⁹.

The crystallinity index of Avicel was monitored via X-ray diffraction during its hydrolysis by a commercial mixture of cellulases from Trichoderma reesei and an excess of β-glucosidase (to prevent cellobiose inhibition). The degree of crystallinity of Avicel was estimated to be 92% using the peak intensity method (which already may be subject to argument, as NMR gives an average of 60% and other X-ray diffraction results reported values between 80 and 90%, but it gives numbers for relative comparison, see above), and this index was found to fluctuate by +/−3% (data not shown). Since the absolute values are already high, a change in crystallinity might be difficult to monitor and interpret using this technique; therefore, CP/MAS ¹³C-NMR spectroscopy was employed as alternate method. The crystallinity index of untreated Avicel (calculated according to published method²⁸) averaged 61% and was found to be constant over the course of hydrolysis, until ca. 90% conversion. No significant change could be monitored (FIG. 4). The slight fluctuations observed around this average were attributed to experimental errors.

Thus, the current thinking according to which amorphous parts of cellulose get hydrolyzed first is not consistent with these results and cannot account for the sharp decrease in rate observed along the reaction. A number of studies that reported an increasing crystallinity along enzymatic hydrolysis attributed the rate slowdown to this crystallinity change^{35, 42, 66-68}. However, changes reported were often modest. Based on the results presented in this work on the relationship between crystallinity and hydrolysis rate, it does not seem physically possible that a change by some percentage points results in such dramatic drops in rate (FIG. 3 shows that a 10% increase in CrI at high CrI values leads to a 40% decrease in initial rate). Therefore, the finding that crystallinity remains constant over enzymatic hydrolysis needs to be eventually considered as definitive for pure cellulosic substrates. Other factors impeding enzymatic action, both enzyme- and substrate-related, need closer attention.

Similarly, using purified cellobiohydrolase I Cel7A from Trichoderma reesei (FIG. 5), instead of a mixture of cellulases, no crystallinity change was observed, however, variations in relative peak intensity in X-ray diffraction patterns showed that Cel7A attacked preferentially the (021) plane of the crystal, as the peak corresponding to this face (centered around 21)° disappeared already after 20% conversion (FIG. 6). Overall, peak intensity ratios for the other peaks were conserved (planes (101), (10 1̅), (002) and (040) at 15, 16, 22.5 and 35°, resp.). The same trend was observed with the commercial cellulases mixture, implying no competition for this plane from the other enzymes (endoglucanases, cellobiohydrolase II and β-glucosidase) or a stronger behavior from. Cel7A. The implications of this preferential attack need to be further investigated as this may provide options to engineer Cel7A and enables overall faster hydrolysis

### Adsorption

There are multiple substrate-related factors that can influence the reaction rate in the enzymatic hydrolysis of cellulose (cf. introduction). With the results presented in this work on the determining role of crystallinity in the enzymatic activity, it is logical to wonder whether crystallinity is simply not hiding another phenomenon, specifically adsorption. Adsorption studies were therefore conducted using cellulose samples generated with various amounts of phosphoric acid and thus displaying intermediate degrees of crystallinity. Adsorption experiments were carried out at 4° C. to prevent the hydrolysis of cellulose and the resulting loss of adsorbent material that would ultimately bias the results. Also, adsorption profile at 4° C. was found to be similar to that at 50° C. after 30 min⁴⁴. The adsorption step has been shown to be fast with half of the maximally adsorbed enzyme bound with 1-2 min and adsorption equilibrium reached after 30 min²².

Adsorption experiments were first performed at reaction loading (amount of enzyme used to generate hydrolysis rates in FIGS. 1-3, i.e. 175 μg/mg cellulose). Surprisingly, a maximum value of adsorbed enzymes (ca. 150 μg/mg cellulose) was reached for cellulose samples with a crystallinity index below a threshold value of ca. 45% (FIG. 7a, open triangles), whereas the amount of adsorbed enzymes seemed to increase inversely and linearly with the crystallinity index at higher crystallinity values (i.e. >45%). A constant amount of adsorbed enzymes (ca. 150 μg/mg cellulose) led to faster hydrolysis reaction at lower degrees of crystallinity (i.e. <45% CrI, FIG. 7b), proving the determining role of crystallinity index in cellulose reactivity. This is most likely due to a difference in the amount of productively bound enzymes, and percentage of surface coverage. Indeed, at low degrees of crystallinity, adsorbed enzymes are being more active at same concentration (i.e. initial rates are higher, FIG. 7c), probably due to the cellulose structure being more open, and therefore preventing enzymes sitting on neighboring chains to hinder one another⁶⁹. At very low crystallinity indexes and same adsorbed enzymes concentration, percentage of surface coverage is different because surface area is larger at lower crystallinity. Exoglucanases may also have higher chances to locate a chain end on an open structure and thus be able to start hydrolysis right away upon binding (initial rates were determined after only 2 min reaction time). Accessibility has been presented as an important factor affecting enzymatic hydrolysis rates⁶⁴. It has been also suggested that making the substrate more amorphous increased access to the reducing-ends of cellulose, thus enhancing rates⁵¹. These data partially supports that hypothesis but importantly, demonstrates that the effect of improved access on hydrolysis rate is limited to higher degrees of crystallinity, while at low degrees of crystallinity, rate enhancement is strictly due to dynamic cause (independently of the adsorption phase). This can be related to recent work showing that overcrowding of enzymes on cellulose surface lowers their activity⁷⁰.

At higher enzyme loading (7 times the original loading, i.e. 1230 μg/mg cellulose), initial rates were found to be generally higher (FIG. 7c, filled circles), confirming published works^{22, 39-42}, but that trend was especially true at lower degrees of crystallinity. Untreated Avicel (CrI=60%) displayed indeed similar rates at both enzyme concentrations, showing that all hydrolysable fractions of cellulose were already covered by enzymes at lower loading, despite different amount of cellulases being adsorbed (high enzyme loading resulted in saturated Avicel while low enzyme loading led to more than half-saturation, binding isotherms not shown). In other words, a higher cellulose surface coverage (in an under-saturated regime) does not necessarily lead to higher rates. The role of adsorption for a given cellulose sample appears to be more crucial to the enzymatic rate at lower degrees of crystallinity (cf. grey shaded area, FIG. 7c).

At higher enzyme loading, crystallinity seems to play a minor role (FIG. 7a). Despite scattered data (due to experimental variations coming from viscous enzymatic solution and large volume added), there is a larger range of degrees of crystallinity were the amount of adsorbed enzymes linearly increases (CrI between 60 and 35%). The breakpoint can be estimated to be around 35% (compared to 45% at lower enzyme loading). Below 35% CrI, a maximum of absorbed cellulases was reached (ca. 600 μg/mg cellulose), while initial rates were still increasing (FIGS. 7a-b), highlighting also the crucial role of crystallinity in enzymatic action at higher enzyme loading. The breakpoint under which crystallinity is the only determining rate factor is expected to decrease as enzyme loading increases, as it gets comparatively harder to reach maximum binding capacity (saturation) at low degrees of crystallinity (open cellulose structure), and also maximum coverage of hydrolysable fractions (investigations underway).

### Example 2

### Fibrous Cellulose

Example 1 was repeated in substantially the same manner except that fibrous cellulose was substituted for Avicel and results are shown in, for example, FIG. 8b.

### Example 3

### Lignocellulosic Material

Example 1 may be repeated substituting a lignocellulosic material for Avicel and similar results are expected. As one skilled in the art will realize, the presence of lignin may inhibit or slow hydrolysis. Accordingly, additional data points may be beneficial when taking measurements of, for example, hydrolysis rate.

### CONCLUSIONS

In summary, the direct relationship between cellulose crystallinity and enzymatic hydrolysis rate was demonstrated on pure cellulose samples (Avicel) and fibrous cellulose. FIG. 8a shows the calculated crystallinity vs. enzymatic hydrolysis rate for Avicel while FIG. 8b shows the calculated crystallinity vs. enzymatic hydrolysis rate for fibrous cellulose. In both FIGS. 8a and 8b the hydrolysis rates correspond to the amount of glucose produced in the first 2 min of the reaction with cellulases. The linear equations shown are the fits between degrees of crystallinity calculated with whole spectra and hydrolysis rates.

The use of a convenient method to reach intermediate degrees of crystallinity by varying the phosphoric acid concentration in the pretreatment step allows the exclusion of any additional parameter that might influence the enzymatic action on cellulose, such as type and source of cellulose or mixed components, and finally yields explicit proof of the tight relationship between initial cellulose crystallinity and rate of degradation by cellulases from Trichoderma reesei. Adsorption capacity, though influencing rate at high degrees of crystallinity and in under-saturated regime, was shown to be constant at lower degrees of crystallinity despite increasing rates, even more so at low cellulose coverage. No change on the crystallinity of cellulose over the course of the enzymatic degradation could be detected, pointing at other reasons for rates slowdown and clearly contradicting the widespread current view according to which mixed cellulose samples would get their amorphous components hydrolyzed first. As crystallinity data interpretation is not trivial, looking at initial hydrolysis rates may be an elegant alternative to determine the degree of crystallinity of pure cellulose. Constant crystallinity and decreased rates point at surface changes occurring with cellulose rapidly after the beginning of the hydrolysis. Specific features of the enzymatic mode of hydrolysis were discovered via X-ray diffraction: cellobiohydrolase I (Cel7A) preferentially attacks the (021) plane of the cellulose crystal. Despite their ability to distinguish different degrees of crystallinity, cellulases are not efficient at reducing/disrupting cellulose crystallinity, most likely due to the fact that cellulose chains are hydrolyzed as soon as their interactions with the crystal are disrupted, leaving therefore an overall unchanged crystallinity but a structure reduced in size.

Future trends for applications of cellulases in the biofuel technology should therefore focus on efficient ways to disrupt cellulose crystallinity and render the overall process economically more viable by reducing the time required to reach full conversion.

### Example 4

### Using Initial Hydrolysis Rate to Improve Efficiency of Enzymatic Hydrolysis

As an example, a calculation of the effort to hydrolyze fibrous cellulose (FC) from cotton linters may be reviewed. If a hydrolysis rate of 5 mg glucose/mL is measured over the first 2 minutes at pH 5 and 50° C. with a cellulose concentration of 20 g/L, the correlation of FIGS. 7, 8a, and 8b yields a degree of crystallinity of 39.4%. If 15 Units per mL of reacting solution of beta-glucosidase and 3.8 FPU (filter paper units) per mL of reacting solution of cellulases (Celluclast® from Novozymes) are used in the hydrolysis of the cellulose in question, then the initial hydrolysis rate of larger batches of the same cellulose will again be 5 mg of glucose per mL of reacting solution.

### Example 5

### Severity of Treatment of Loblolly Pine (LP)

The present example focuses on the effect of severity on digestibility, accessibility, and crystallinity index (CrI) of Loblolly pine (LP) softwood; the dominant tree species in southeastern United States. A simple method was utilized to accurately and quickly determine the CrI of both untreated and steam-exploded lignocellulosic biomass from X-ray diffraction (XRD) spectra.

### Materials

Avicel PH-101 (11365) purchased from Sigma-Aldrich (St Louis, Mo., USA) was employed as standard crystalline cellulose in this study. Three differently-pretreated SELP samples were generously provided by Dr. John Muzzy from the Georgia Institute of Technology, Atlanta, Ga., USA. Cellulase from Trichoderma reesei (Celluclast®, 159 FPU/ml) and β-glucosidase from almonds (5.2 U/mg) were purchased from Sigma-Aldrich and phosphoric acid (85%) was from EMD (Gibbstown, N.J., USA). The BCA protein assay kit was purchased from Thermo Fischer Scientific (Rockford, Ill., USA).

### Sample Preparation

The acidic SELP samples as received were extensively washed with DI water and sodium acetate (NaOAc) buffer (50 mM, pH 5.0) until pH 5.0 was reached. Lyophilization at −55° C. and 0.05 mbar was used to dry the washed samples overnight, and the absence of soluble reducing sugars in lyophilized samples was confirmed by dinitrosalicylic acid (DNS) assay.

### Enzymatic Hydrolysis

Batch hydrolysis reactions were carried out in 1.5 ml eppendorf tubes with 2% substrate consistency. Substrates were preincubated for 1 h in NaOAc buffer (50 mM, pH 5.0) at 45° C. with stirring. After preincubation, enzymatic hydrolysis was carried out for a desired time with varying enzyme loadings at the same conditions. β-Glucosidase was added at 15 kU/L. The total reaction volume was 1 ml unless otherwise stated. At the desired time point, the tubes containing reaction mixture were immediately chilled on ice and centrifuged at 14,000 rpm for 3 min. The reducing sugar concentration in supernatant was measured by DNS assay with glucose standards.

### Steam Explosion

LP sample was mixed with the desired concentration of gaseous SO₂ for at least 1 hour in a sealed plastic bag. The material was then pretreated with steam in a 10-L pretreatment reactor at the desired temperature for a specified residence time. After steam pretreatment, a pneumatic ball valve was opened rapidly and the contents were explosively discharged into a 100-L accumulator at atmospheric pressure. Solid residue was collected from the accumulator and stored at −20° C. for further analysis.

### Adsorption Studies

Substrates (2% consistency) were preincubated in sodium acetate buffer (50 mM, pH 5.0) at 45° C., 1000 rpm for 1 h followed by cooling on ice. Varying amounts of cellulase were added and further agitated for 30 min at 4° C. to minimize the influence from hydrolysis. Following 30 min adsorption, all samples were immediately centrifuged for 3 min at 4° C. and 18,625 g.

### Protein Assay

A modified BCA assay with precipitation was utilized to determine protein concentrations in the supernatant after cellulase adsorption. Control experiments lacked substrate. This procedure has been previously published by R. Brown et al. (Brown et al., 1989). The adsorbed (bound) cellulase was calculated as the total enzyme added minus free enzyme in the supernatant.

### X-Ray Diffraction (XRD)

XRD patterns of lyophilized samples were recorded with a X′ pert PRO X-ray diffractometer (PANanalytical BV, Almelo, the Netherlands) using Cu/Kα₁ irradiation (1.54 Å) at 45 kV and 40 mA. The scattering angle (2θ) ranged from 10° to 40° with a scan speed 0.021425 s⁻¹ and step size 0.0167°.

### Phosphoric Acid (PA) Treatment

PA treatment of steam-exploded Loblolly pine (SELP) were carried out following the procedure described by M. Hall et al (Hall et al., 2010). Thirty mL of diluted (0° C.) PA was used to pretreat 1 g slightly hydrated SELP for 40 min with occational stirring. Thirty ml of acetone (0° C.) was used for regeneration of cellulose. The resulting solid was washed twice with 30 ml acetone (0° C.), and four times with 100 ml DI water. The washed solid fraction was used for initial rate experiments directly, and the lyophilized powder was prepared for XRD measurement.

### Determination of Acid-Insoluble Lignin (AIL) Content

The AIL content in SELPs was measured by dilute sulfuric acid hydrolysis. Samples of 175 mg oven-dried SELPs were first swelled in 1.5 ml 72% H₂SO₄ for 4 h with occasional stirring. The mixture was then diluted to 42 ml and autoclaved at 121° C. for 1 h. After cooling to room temperature, the mixture was vacuum dried for 20 min, and the insoluble solids were further dried at 105±3° C. in an oven overnight. The AIL content was calculated based on the mass balance.

### Results and Discussion

The goal of this study was to determine how the severity of steam explosion pretreatment can affect the digestibility and accessibility of pretreated lignocellulosics, and the interaction between cellulose and lignin. Furthermore, the significance of CrI in the enzymatic hydrolysis of pretreated lignocellulosics was elucidated.

### Hydrolysis of SELPs

Steam explosion is considered to be one of the most effective and relatively low-cost pretreatment methods for hardwood as well as softwood (Sudo et al., 1986). The additional step of SO₂ impregnation aims for an improved recovery of both cellulose and hemicelluloses (Taherzadeh & Karimi, 2008). After pretreatment, the solid biomass residue contains most of cellulose and lignin, while the majority of hemicellulose is degraded and solubilized into the liquid biomass fraction.

In this work, the effects of steam explosion on the solid residue are investigated. Three SELPs under different pretreatment severities were extensively washed with DI water and sodium acetate buffer to adjust the pH value to 5.0 and remove soluble substances and inhibitors. The samples were designated as SELP 1, SELP 2 and SELP 3 in order of ascending severity. The pretreatment conditions and resulting severities are shown in Table 1 below.

TABLE 1

| | SO2 | Temperature | Residence time | Severity |
|---|---|---|---|---|
| | (%) | T (° F.) | t (min) | R |
| SELP 1 | 2.9 | 419 | 5 | 3.1 |
| SELP 2 | 5 | 417 | 5 | 3.2 |
| SELP 3 | 3 | 435 | 10 | 3.6 |

The severity index (R) was calculated according to the following Equation 1 (Nguyen et al., 2000):

$$R = {log}_{10} ( t {e}^{\frac{T - 100}{14.75}} ) - pH \qquad ( 1 )$$

where t is residence time (min), T is temperature (° C.).

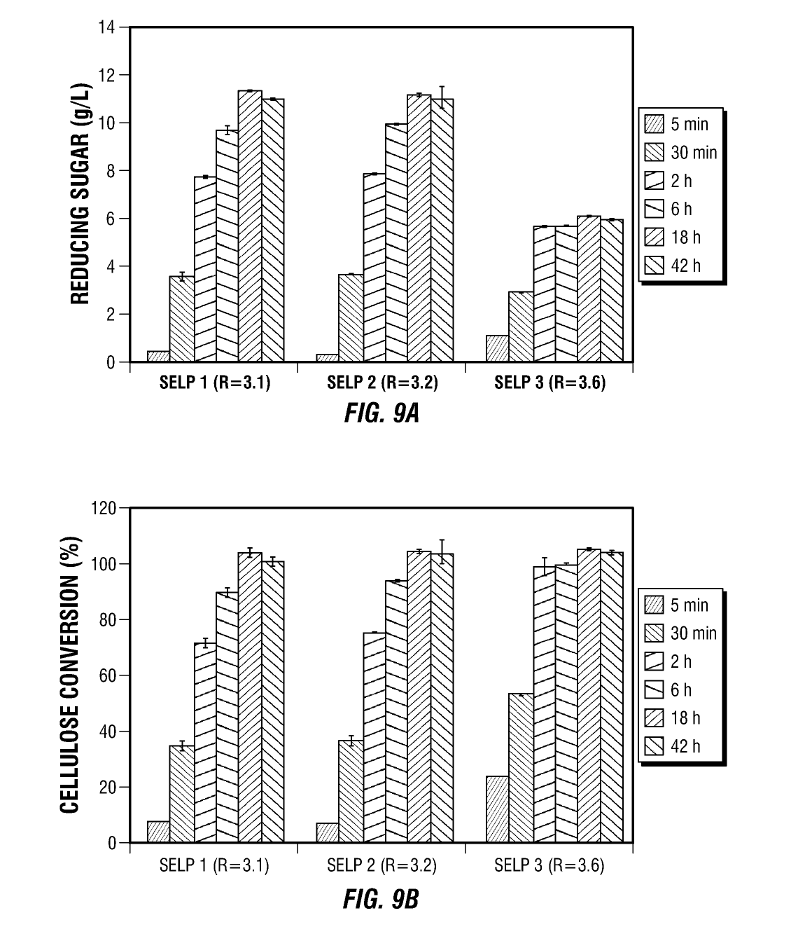

FIG. 9a shows the histogram of a 42 h hydrolysis for all SELP samples. SELP 3 has the lowest absolute concentration of reducing sugars after 42 h degradation, which is indicative of low remaining hydrolysable cellulose content. The degree of cellulose hydrolysis after enzymatic degradation was calculated to be 60% for both SELP 1 and 2, while only 32% for SELP 3. As the severity increases, cellulose is hydrolyzed and solubilized into the liquid fraction, thus increasing the percentage of lignin in the solid residue. After normalization to total cellulose content (FIG. 9b), SELP 3 shows the highest sugar concentration among the three samples, despite containing a much lower cellulose content to begin with. Solubilized sugar concentration in hydrolysate of SELP 1 and 2 are similar throughout the 42 hour period. The sugar concentration saturated by 18 h for SELP 1 and 2, while the saturation was achieved much earlier at around 2 h for SELP 3. Thus, there is a compromise between the pretreatment selectivity, which measures the relative removal of hemicelluloses and cellulose during steam explosion, and resulting cellulose hydrolysability. High steam explosion severity results in a more degradable cellulose fraction, while the high selectivity of SO₂-catalyzed acid hydrolysis during pretreatment is not maintained (Ramos, 2003). In addition to the decreased cellulose concentration, the presence of lignin will inhibit the enzymatic reaction by two mechanisms: forming a physical barrier which reduces the accessibility of cellulose and non-productive binding with cellulosic enzymes to impair their efficiency (Zhu et al., 2010). Thus, the combined but countervailing effects of increased lignin content and enhanced cellulose accessibility determine the hydrolysis rate and extent of differently steam-pretreated LP.

### Adsorption and Initial Rate Studies

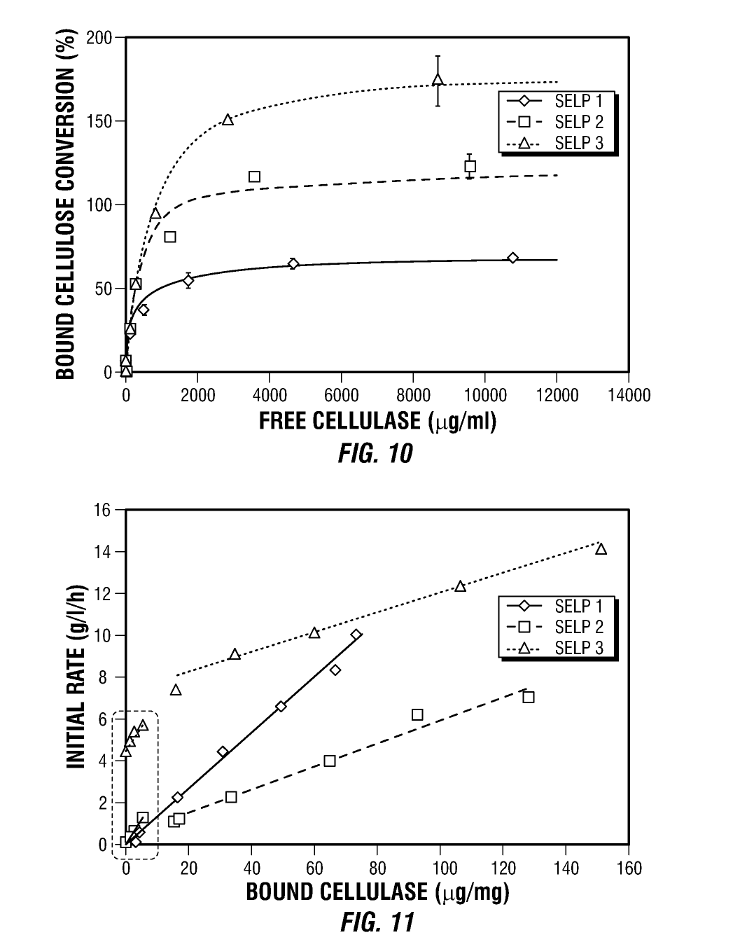

Due to the characteristics of both the substrates and enzymes, adsorption is a prerequisite step for the catalytic reaction to occur. The adsorption isotherm was obtained at 4° C. to minimize carbohydrate degradation during adsorption. Carbohydrate degradation reduces the number of available active binding sites and introduces error in adsorption studies (Hall et al., 2010). The adsorption of cellulolytic enzyme onto substrates is an effective process which usually requires 2-5 min to achieve equilibrium; 30 min was allowed in this study for equilibrium to establish (Dijkerman et al., 1996; Tsao, 1999). FIG. 10 shows the SELPs adsorption isotherms. The substrate accessibility can be calculated using the adsorbed cellulase (the difference between total enzyme loaded and free enzyme remaining in supernatant). The experimental adsorption data was fitted to Langmuir isotherm having the following form:

$$B = {B}_{ma x} \frac{ {K}_{ad} C}{1 + {K}_{ad} C} \qquad ( 2 )$$

where B is the adsorbed cellulase amount (μg/mg substrate), Bₘₐₓ is the maximum adsorption amount (μg/mg substrate), K_{ad} is adsorption equilibrium constant (l/g), and C is free cellulase concentration (g/l).

All three SELPs have similar affinity to cellulase enzymes at low enzyme loadings (<0.78 mg/ml); noticeable differences begin to appear beyond 0.78 mg/ml. From the nonlinear fitting results, cellulase adsorption on all SELPs tested follows Langmuir isotherm with R²=0.971 or higher. The parameters Bₘₐₓ and K_{ad} determined from each Langmuir isotherm are listed in Table 2 below.

TABLE 2

| | R² | Bₘₐₓ | K_{ad} |
|---|---|---|---|
| | (%) | (μg/mg) | (ml/mg) |
| SELP 1 | 99.6 | 75.7 | 0.005 |
| SELP 2 | 97.1 | 132.3 | 0.0037 |
| SELP 3 | 99.6 | 184.4 | 0.0016 |

Among the three samples, SELP 3 showed the highest Bₘₐₓ value of 184.4 mg/g, which indicates the highest accessibility to enzyme. Lu et al. studied the adsorption behavior of steam-exploded Douglas-fir, (which can also be fit to a Langmuir isotherm) and found the maximum adsorption capacity to be 171.3 mg/g substrate (Lu et al., 2002). The relative magnitude of accessibility of all three substrates investigated are SELP 3>SELP 2>SELP 1. The accessibility of substrates is positively correlated with the pretreatment severity.

The binding capacity is a reflection of the accessible substrate surface which includes cellulose, lignin and trace amounts of hemicellulose. Binding capacity can be ascribed to cellulase adsorption on both cellulose and lignin, which has been reported to have a high affinity toward cellulolytic enzymes (Nakagame et al., 2011; Tu et al., 2009). There is an apparent difference in the adsorption capacity of SELP 1 and 2, which likely resulted from the different SO₂ concentrations used for LP impregnation prior to pretreatment. A higher SO₂ concentration of 5% in SELP 2 promoted removal of hemicellulose, and increased lignin mobility after reaching its softening temperature around 200° C. (Gellerstedt, 2008; Yin et al., 2007). The softening and redistribution rendered lignin more accessible to cellulolytic enzymes, which contributes mainly to non-productive binding. No obvious difference in adsorption will be observed unless high enzyme loadings are employed, at which point the available active sites on the substrate become limiting. The subsequent initial rate experiment plots initial rate as a function of total adsorption capacity (FIG. 11). A wide range of enzyme concentrations (0.0039-6.24 mg/ml) was tested and a linear relationship was found for SELP 1 throughout the range. As for SELP 2 and 3, a “phase transition” was observed, which is indicative of a substrate reactivity change over the enzyme concentration range. At low enzyme loadings (those boxed in FIG. 11), the slope of SELP 3 is the highest, followed by SELP 2; this phenomenon is probably due to the difference in cellulose morphology after steam explosion. To the contrary, at higher enzyme loadings, the slope order is SELP 1>SELP 2>SELP 3 (0.1361, 0.0575, 0.0504, respectively); the reversed order can be explained by the limited number of available binding sites on reactive fraction of the substrate as enzyme loading increases. An increasing portion of bound cellulase ended up non-productively bound onto lignin. The slope is an indicator of the reactivity of substrate; a smaller slope implies more cellulase adsorption is required to obtain same initial rate at the enzyme concentrations investigated. Although both SELP 1 and SELP 2 show similar initial rates, the substrate affinity is markedly different. SELP 2 is more accessible to cellulase compared to SELP 1, while the cellulase adsorption on SELP 1 is much more productive than SELP 2 due to a similar amount of reducing sugar released (FIG. 9a) despite the difference in adsorption capacity. The difference in initial rate between SELP 3 and the other two SELPs is more evident at relatively low enzyme loadings because of its exceptional high reactivity at extremely low enzyme concentration, which is not easy to capture. Significant lignin and/or cellulose structural change, which resulted in distinct adsorption behavior, must have occurred for the three SELPs during the process of steam explosion.

### CrI Measurement of SELPs

The determination of CrI for the cellulose fraction in pretreated wood samples can be tricky because of the existence of many interfering factors such as the presence of lignin, hemicellulose, residual extractives, and the interaction between them. To quantitatively determine CrI, a simple mathematical method, Least-Squares Estimation (LSE), was utilized to estimate CrI with reasonable accuracy for complicated lignocellulosic substrates.

Due to the ease of use, about 70-85% published studies calculated the CrI of cellulose/lignocellulosics based on the peak height method; this method was developed in 1962 by L. Segal et al. as an empirical method to obtain CrI from XRD pattern (Park et al., 2010; Segal L, 1962). From the semi-quantitative peak height method which calculates the CrI based on the relative reflected intensity of the 200 plane and amorphous cellulose, one can easily ascertain the relative CrI of those samples. However, to attain a more quantitative result, the whole XRD spectrum needs to be taken into account to minimize loss of information. The normalized spectrum of a sample is expressed as a linear combination of the normalized spectra of crystalline cellulose, amorphous cellulose, and lignin (Andersson et al., 2003; Bansal et al., 2010). Since a large portion of hemicellulose was dissolved and removed by washing steps, the remaining trace amount was ignored in the calculation of CrI. Equation 3 shows the linear combination:

I(2θ)=fₐIₐ(2θ)+fₗIₗ(2θ)+f_{c}I_{c}(2θ)+ε (3)

fₐ+fₗ+f_{c}=1 (4)

where Iₐ(2θ) is the intensity of amorphous cellulose at 2θ, Iₗ(2θ) is the intensity of lignin at 20, I_{c}(2θ) is the intensity of crystalline cellulose at 2θ, fₐ is the contribution of amorphous cellulose to the spectra, fₗ is the contribution of lignin, f_{c} is the contribution of crystalline cellulose, and ε is random error. The summation of the above three contributors approximately equals 1 (Equation 4).

Model crystalline cellulose (Avicel), amorphous cellulose (phosphoric acid swollen cellulose or PASC), and commercial lignin are used as standards. LSE was performed to estimate the two parameters fₐ and fₗ with the sample spectrum and the standards. The accuracy of the curve-fitting was evaluated via R²; comparing how well the original spectra are likely to be predicted by the reconstructed spectra (Table 3). Since neither the amorphous cellulose nor lignin contains crystalline structure, the CrI was calculated by Equation 5:

CrI=(1−fₐ−fₗ)CrI_{c} (5)

where CrI_{c} is the known CrI of Avicel which is determined by ¹³C-NMR to be 60%.

The curve fitting results are shown in Table 3 below.

TABLE 3

| | CrI | R² | AIL | CrI_{cel} |
|---|---|---|---|---|
| | (%) | (%) | (%) | (%) |
| LP | 26.2 | 0.98 | — | 58.2 |
| SELP 1 | 41.9 | 0.98 | 33.9 | 63.4 |
| SELP 2 | 31.2 | 0.97 | 39.7 | 51.7 |
| SELP 3 | 12.9 | 0.89 | 66.7 | 38.7 |

Table 3 leads to the conclusion that the overall CrI of SELPs decreases with rising pretreatment severity. The decrease in CrI is a combined result of the removal of amorphous components and the structural change of cellulose crystal. Furthermore, the cellulose CrI, which is of more interest to the community, can be calculated based on the linear combination assumption with Equation 6 (results shown in Table 3).

$${CrI}_{cel} = \frac{CrI}{{F}_{c}} , \qquad ( 6 )$$

where CrI_{cel} is the CrI of cellulose fraction in SELPs, F_{c} is the percentage content of cellulose in the sample. The change in CrI_{cel} shows the same trend as the overall CrI of SELPs. The untreated LP contains 45% cellulose (Zhu & Pan, 2010), and the CrI_{cel} was calculated using Equation 6 to be 58.2%. Compared to untreated LP, SELP 1 (the lowest severity) showed an increase in the cellulose CrI. Further increases in severity resulted in a decrease of cellulose CrI in LP samples. The change in CrI from untreated LP to SELP 1 is in agreement with most published results (Cheman et al., 2010; T. Yamashiki, 1990) and the increase can be ascribed to the preferential hydrolysis of amorphous cellulose during steam explosion (Wang et al., 2009).

The finding that further increase in severity generates a LP sample with more amorphous-like cellulose is surprising and unexpected. Without wishing to be bound to any particular theory, it is believed that the decrystallization effect of steam explosion occurs at a certain severity and may be accompanied by nonselective carbohydrate degradation. At low severity, the increase in CrI is mainly due to the hydrolysis of amorphous fraction including hemicelluloses and possibly a small amount of amorphous cellulose. As severity further increases, the effect of steam explosion will not be limited to amorphous region but also disrupt the crystalline portion. Some researchers isolated cellulose from steam exploded agricultural residues by alkali extraction and bleaching to also find a decrease in CrI as severity increases (M. Ibrahim, 2010).

In addition, the effect of steam explosion on pure cellulose isolated from wood and cotton was studied, and the same trend was confirmed (T. Yamashiki, 1990). The effect of steam explosion on isolated pure cellulose and cellulose which is embedded in a complex plant polymers matrix will likely differ; moreover, previously published work (T. Yamashiki, 1990) did not investigate a severity high enough to be comparable with this study's SELP 3. To assess the actual impact of steam explosion on substrate CrI, the possibility that additional isolation/extraction steps introduce further CrI and/or cellulose crystal structure change requires caution when choosing extraction conditions. By studying steam explosion severity from low to extremely high levels and avoiding additional isolation steps of biomass constituents, the true effect of steam explosion itself can be revealed. The results from this work provide evidence that there exists a critical severity point below which the CrI_{cel} shows positive correlation with severity while the opposite is true when exceeding this point.

### Phosphoric Acid Treatment of SELP

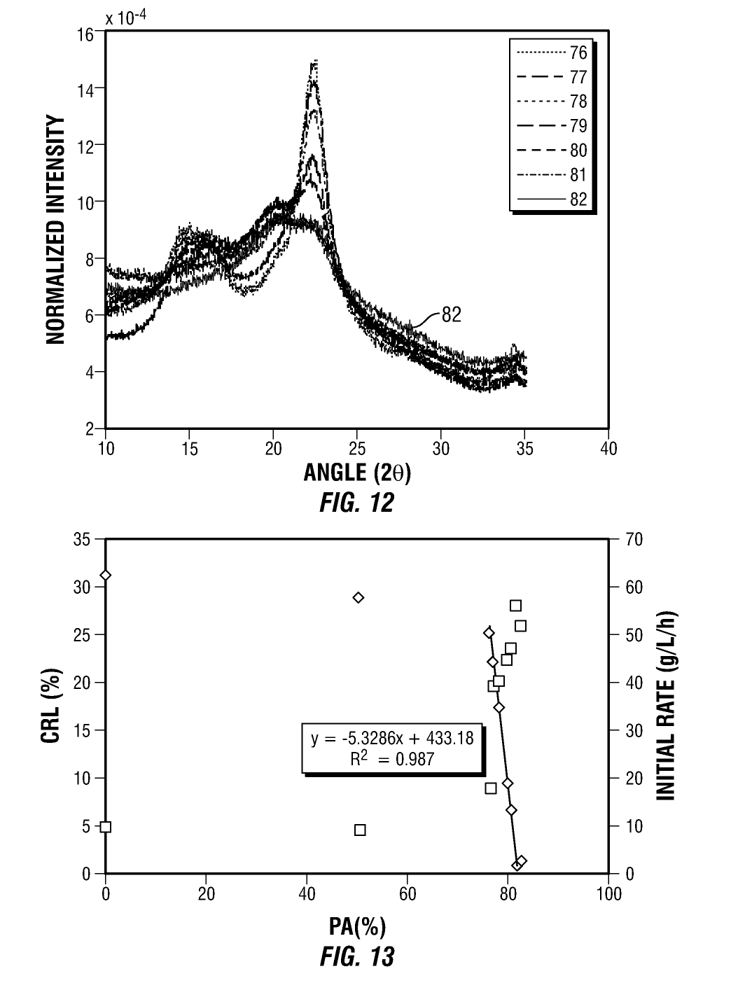

The CrI of pure cellulosic substrate (e.g. Avicel) is a crucial factor in the hydrolysis process (Fan et al., 1980; Hall et al., 2010; Taherzadeh & Karimi, 2008). To investigate the role of CrI in the hydrolysis rate of pretreated lignocellulosics rather than Avicel, the interference from compositional difference needs to be ruled out. Thus, phosphoric acid (PA) treatment was performed on SELP 2, and its crystalline structure was analyzed via X-ray diffractometry before and after PA treatment. PA has been used as a standard method to create amorphous cellulose with a high level of accessibility to enzymes (Den Haan et al., 2007). The XRD spectra of PA-treated SELP 2 (PASELP) are shown in FIG. 12. A series of PA concentrations ranging from 76% to 82% were employed to generate PASELPs with different CrI, calculated by the above-mentioned curve fitting method. It is observed from FIG. 12 that PA preferentially attacks the crystal planes 200 and 110; a sufficiently concentrated PA can completely destroy those two planes resulting in a completely amorphous SELP substrate. As PA concentration increases from 76% to 82%, the CrI of the resulting PASELP decreases linearly (diamond points in FIG. 13). The CrI of PASELP is quite sensitive to PA concentration change and only a narrow range has detectable impact on CrI. PA of a concentration lower than 76% is not capable of decrystallizing steam-pretreated lignocellulosics. The presence of critical PA concentrations was also reported by previous studies to be 75% and 83% for Avicel and industrial hemp, respectively (Hall et al., 2010; Moxley et al., 2008).

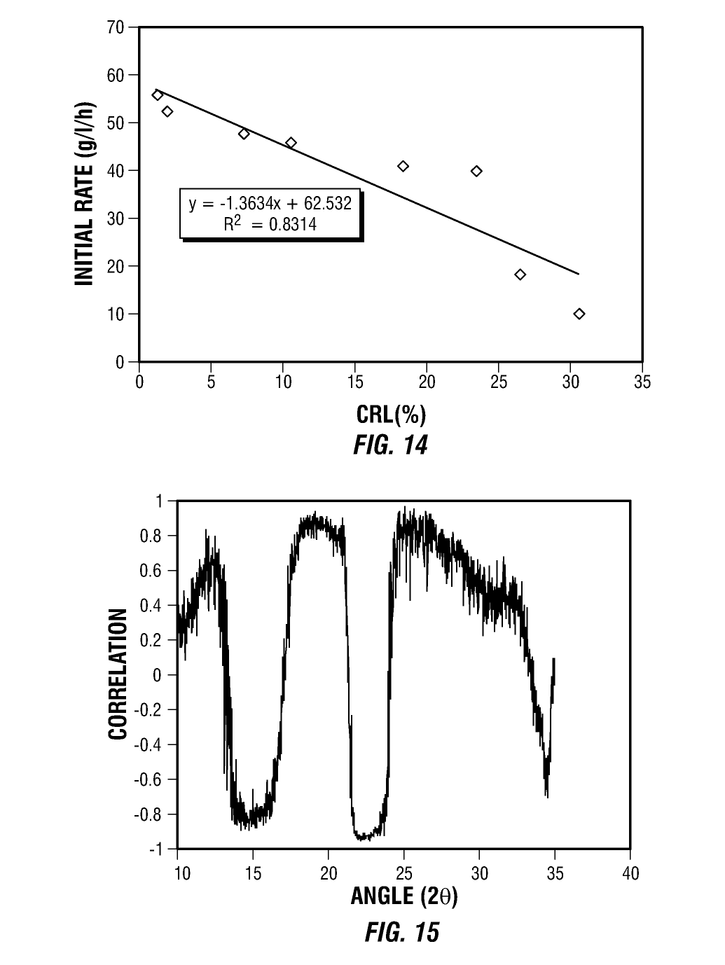

Initial rate experiments were carried out to determine the hydrolysability of PASELP. A CrI change of ˜30%-points increases the initial rate fivefold (square points in FIG. 13) and a linear relationship was found between initial rate and CrI (FIG. 14). Although CrI is always coupled with other properties of cellulosic/lignocellulosic biomass (Park et al., 2010) such as accessibility (P. Bansal)accepted). The calculation of CrI is independent of other properties and is capable of excluding any interference from other factors. The linear dependence of initial hydrolysis rates on independently calculated CrI enables us to conclude that CrI plays an important role in pretreated lignocellulosics.

Furthermore, the correlation between initial rates and intensities was analyzed at all diffraction angles ranging from 10° to 35° in seven differently treated PASELPs. The correlation plot is shown in FIG. 15. There is strong negative correlation (>0.85) at diffraction angles around 110 and 200 phases, at 2θ=15° and 22.5° respectively, which means the decrement of intensities in those regions has a positive contribution to the initial rate of samples. Although no physical meaning has been assigned to the interval between these two phases, the intensities were found to correlate positively with hydrolysis rates. This range of angles almost superimposes with that of the location of peaks of amorphous cellulose, thus, the hydrolysis rate increases with increased amorphous fraction in samples can well explain the resulting correlation in this region. The results shown in FIG. 15 are in good agreement with previous findings for Avicel (Bansal et al., 2010).

### CONCLUSIONS

The present example focused on the effects of severity on the steam explosion of Loblolly pine (LP). It is evident that the steam explosion process at low severity preferentially removes the amorphous constituents, resulting in an increase in the CrI of SELP. However, further increases in severity generate a substrate with lower CrI; a combined effect of the removal of amorphous components and disruption of crystal structure. A critical severity point exists in steam explosion beyond which the degradation of crystalline cellulose dominates the process rather than the opening of a tight cellulose-lignin substructure. The significance of CrI in enzymatic hydrolysis of pretreated lignocellulosics was confirmed by a linear dependency of initial rate on CrI. This finding leads us to believe that CrI is an indicator of hydrolysis rate and digestibility of pretreated lignocellulosics.

The claimed subject matter is not to be limited in scope by the specific embodiments described herein. Indeed, various modifications of the invention in addition to those described herein will become apparent to those skilled in the art from the foregoing description. Such modifications are intended to fall within the scope of the appended claims.

The citation of any publication is for its disclosure prior to the filing date and should not be construed as an admission that the present invention is not entitled to antedate such publication by virtue of prior invention and/or priority applications.

### REFERENCES

1. Himmel, M. E.; Ding, S. Y.; Johnson, D. K.; Adney, W. S.; Nimlos, M. R.; Brady, J. W.; Foust, T. D., Biomass recalcitrance: Engineering plants and enzymes for biofuels production. Science 2007, 315, (5813), 804-807.
2. Himmel, M. E.; Ruth, M. F.; Wyman, C. E., Cellulose for commodity products from cellulosic biomass. Current Opinion in Biotechnology 1999, 10, (4), 358-364.
3. Wyman, C. E., What is (and is not) vital to advancing cellulosic ethanol. Trends in Biotechnology 2007, 25, (4), 153-157.
4. Lynd, L. R.; Laser, M. S.; Brandsby, D.; Dale, B. E.; Davison, B.; Hamilton, R.; Himmel, M.; Keller, M.; McMillan, J. D.; Sheehan, J.; Wyman, C. E., How biotech can transform biofuels. Nature Biotechnology 2008, 26, (2), 169-172.
5. Divne, C.; Ståhlberg, J.; Reinikainen, T.; Ruohonen, L.; Pettersson, G.; Knowles, J. K. C.; Teeri, T. T.; Jones, T. A., The 3-Dimensional Crystal-Structure of the Catalytic Core of Cellobiohydrolase-I from Trichoderma-Reesei. Science 1994, 265, (5171), 524-528.
6. Divne, C.; Ståhlberg, J.; Teeri, T. T.; Jones, T. A., High-resolution crystal structures reveal how a cellulose chain is bound in the 50 angstrom long tunnel of cellobiohydrolase I from Trichoderma reesei. Journal of Molecular Biology 1998, 275, (2), 309-325.
7. Teeri, T. T., Crystalline cellulose degradation: New insight into the function of cellobiohydrolases. Trends in Biotechnology 1997, 15, (5), 160-167.
8. Kipper, K.; Väljamäe, P.; Johansson, G., Processive action of cellobiohydrolase Cel7A from Trichoderma reesei is revealed as ‘burst’ kinetics on fluorescent polymeric model substrates. Biochemical Journal 2005, 385, 527-535.
9. Li, Y. C.; Irwin, D. C.; Wilson, D. B., Processivity, substrate binding, and mechanism of cellulose hydrolysis by Thermobifida fusca Cel 9A. Applied and Environmental Microbiology 2007, 73, (10), 3165-3172.
10. Davies, G.; Henrissat, B., Structures and Mechanisms of Glycosyl Hydrolases. Structure 1995, 3, (9), 853-859.
11. Henrissat, B., Cellulases and Their Interaction with Cellulose. Cellulose 1994, 1, (3), 169-196.
12. Knowles, J.; Lehtovaara, P.; Teeri, T., Cellulase Families and Their Genes. Trends in Biotechnology 1987, 5, (9), 255-261.
13. Lynd, L. R.; Weimer, P. J.; van Zyl, W. H.; Pretorius, I. S., Microbial cellulose utilization: Fundamentals and biotechnology. Microbiology and Molecular Biology Reviews 2002, 66, (3), 506-+.
14. Zhang, Y. H. P.; Lynd, L. R., Toward an aggregated understanding of enzymatic hydrolysis of cellulose: Noncomplexed cellulase systems. Biotechnology and Bioengineering 2004, 88, (7), 797-824.
15. Väljamäe, P.; Sild, V.; Nutt, A.; Pettersson, G.; Johansson, G., Acid hydrolosis of bacterial cellulose reveals different modes of synergistic action between cellobiohydrolase I and endoglucanase I. European Journal of Biochemistry 1999, 266, (2), 327-334.
16. Medve, J.; Karlsson, J.; Lee, D.; Tjerneld, F., Hydrolysis of microcrystalline cellulose by cellobiohydrolase I and endoglucanase II from Trichoderma reesei: Adsorption, sugar production pattern, and synergism of the enzymes. Biotechnology and Bioengineering 1998, 59, (5), 621-634.
17. Hoshino, E.; Shiroishi, M.; Amano, Y.; Nomura, M.; Kanda, T., Synergistic actions of exo-type cellulases in the hydrolysis of cellulose with different crystallinities. Journal of Fermentation and Bioengineering 1997, 84, (4), 300-306.
18. Nidetzky, B.; Hayn, M.; Macarron, R.; Steiner, W., Synergism of Trichoderma-Reesei Cellulases While Degrading Different Celluloses. Biotechnology Letters 1993, 15, (1), 71-76.
19. Henrissat, B.; Driguez, H.; Viet, C.; Schulein, M., Synergism of Cellulases from Trichoderma-Reesei in the Degradation of Cellulose. Bio-Technology 1985, 3, (8), 722-726.
20. Bansal, P.; Hall, M.; Realff, M. J.; Lee, J. H.; Bommarius, A. S., Modeling cellulase kinetics on lignocellulosic substrates. Biotechnol Adv 2009, 27, (6), 833-48.
21. Ståhlberg, J.; Johansson, G.; Pettersson, G., A New Model for Enzymatic-Hydrolysis of Cellulose Based on the 2-Domain Structure of Cellobiohydrolase-I. Bio-Technology 1991, 9, (3), 286-290.
22. Steiner, W.; Sattler, W.; Esterbauer, H., Adsorption of Trichoderma-Reesei Cellulase on Cellulose—Experimental-Data and Their Analysis by Different Equations. Biotechnology and Bioengineering 1988, 32, (7), 853-865.
23. Beldman, G.; Voragen, A. G. J.; Rombouts, F. M.; Searlevanleeuwen, M. F.; Pilnik, W., Adsorption and Kinetic-Behavior of Purified Endoglucanases and Exoglucanases from Trichoderma-Viride. Biotechnology and Bioengineering 1987, 30, (2), 251-257.
24. Bader, J.; Bellgardt, K. H.; Singh, A.; Kumar, P. K. R.; Schugerl, K., Modeling and Simulation of Cellulase Adsorption and Recycling during Enzymatic-Hydrolysis of Cellulosic Materials. Bioprocess Engineering 1992, 7, (5), 235-240.
25. Medve, J.; Ståhlberg, J.; Tjerneld, F., Adsorption and Synergism of Cellobiohydrolase-I and Cellobiohydrolase-Ii of Trichoderma-Reesei during Hydrolysis of Microcrystalline Cellulose. Biotechnology and Bioengineering 1994, 44, (9), 1064-1073.
26. Nidetzky, B.; Steiner, W.; Claeyssens, M., Cellulose Hydrolysis by the Cellulases from Trichoderma-Reesei—Adsorptions of 2 Cellobiohydrolases, 2 Endocellulases and Their Core Proteins on Filter-Paper and Their Relation to Hydrolysis. Biochemical Journal 1994, 303, 817-823.
27. Yang, B.; Willies, D. M.; Wyman, C. E., Changes in the enzymatic hydrolysis rate of avicel cellulose with conversion. Biotechnology and Bioengineering 2006, 94, (6), 1122-1128.
28. Bommarius, A. S.; Katona, A.; Cheben, S. E.; Patel, A. S.; Ragauskas, A. J.; Knudson, K.; Pu, Y., Cellulase kinetics as a function of cellulose pretreatment. Metabolic Engineering 2008, 10, (6), 370-381
29. Mansfield, S. D.; Mooney, C.; Saddler, J. N., Substrate and enzyme characteristics that limit cellulose hydrolysis. Biotechnology Progress 1999, 15, (5), 804-816.
30. Xu, F.; Ding, H. S., A new kinetic model for heterogeneous (or spatially confined) enzymatic catalysis: Contributions from the fractal and jamming (overcrowding) effects. Applied Catalysis a-General 2007, 317, (1), 70-81.
31. Zhang, Y. H. P.; Himmel, M. E.; Mielenz, J. R., Outlook for cellulase improvement: Screening and selection strategies. Biotechnology Advances 2006, 24, (5), 452-481.
32. McLean, B. W.; Boraston, A. B.; Brouwer, D.; Sanaie, N.; Fyfe, C. A.; Warren, R. A. J.; Kilburn, D. G.; Haynes, C. A., Carbohydrate-binding modules recognize fine substructures of cellulose. Journal of Biological Chemistry 2002, 277, (52), 50245-50254.
33. Puri, V. P., Effect of Crystallinity and Degree of Polymerization of Cellulose on Enzymatic Saccharification. Biotechnology and Bioengineering 1984, 26, (10), 1219-1222.
34. Fan, L. T.; Lee, Y. H., Kinetic-Studies of Enzymatic-Hydrolysis of Insoluble Cellulose—Derivation of a Mechanistic Kinetic-Model. Biotechnology and Bioengineering 1983, 25, (11), 2707-2733.
35. Ohmine, K.; Ooshima, H.; Harano, Y., Kinetic-Study on Enzymatic-Hydrolysis of Cellulose by Cellulase from Trichoderma-Viride. Biotechnology and Bioengineering 1983, 25, (8), 2041-2053.
36. Fan, L. T.; Lee, Y. H.; Beardmore, D. H., Mechanism of the Enzymatic-Hydrolysis of Cellulose—Effects of Major Structural Features of Cellulose on Enzymatic-Hydrolysis. Biotechnology and Bioengineering 1980, 22, (1), 177-199.
37. Zhu, L.; O'Dwyer, J. P.; Chang, V. S.; Granda, C. B.; Holtzapple, M. T., Structural features affecting biomass enzymatic digestibility. Bioresource Technology 2008, 99, (9), 3817-3828.
38. Berlin, A.; Gilkes, N.; Kurabi, A.; Bura, R.; Tu, M. B.; Kilburn, D.; Saddler, J., Weak lignin-rinding enzymes—A novel approach to improve activity of cellulases for hydrolysis of lignocellulosics. Applied Biochemistry and Biotechnology 2005, 121, 163-170.
39. Sattler, W.; Esterbauer, H.; Glatter, O.; Steiner, W., The Effect of Enzyme Concentration on the Rate of the Hydrolysis of Cellulose. Biotechnology and Bioengineering 1989, 33, (10), 1221-1234.
40. Lee, Y. H.; Fan, L. T., Kinetic-Studies of Enzymatic-Hydrolysis of Insoluble Cellulose—Analysis of the Initial Rates. Biotechnology and Bioengineering 1982, 24, (11), 2383-2406.
41. Ryu, D. D. Y.; Lee, S. B., Enzymatic-Hydrolysis of Cellulose—Determination of Kinetic-Parameters. Chemical Engineering Communications 1986, 45, (1-6), 119-134.
42. Medve, J.; Ståhlberg, J.; Tjerneld, F., Adsorption and Synergism of Cellobiohydrolase-I and Cellobiohydrolase-Ii of Trichoderma-Reesei during Hydrolysis of Microcrystalline Cellulose. Biotechnology and Bioengineering 1994, 44, (9), 1064-1073.
43. Klyosov, A. A.; Mitkevich, O. V.; Sinitsyn, A. P., Role of the Activity and Adsorption of Cellulases in the Efficiency of the Enzymatic-Hydrolysis of Amorphous and Crystalline Cellulose. Biochemistry 1986, 25, (3), 540-542.
44. Lee, S. B.; Shin, H. S.; Ryu, D. D. Y.; Mandels, M., Adsorption of Cellulase on Cellulose—Effect of Physicochemical Properties of Cellulose on Adsorption and Rate of Hydrolysis. Biotechnology and Bioengineering 1982, 24, (10), 2137-2153.
45. Schulein, M., Enzymatic properties of cellulases from Humicola insolens. Journal of Biotechnology 1997, 57, (1-3), 71-81.
46. Wood, T. M., Cellulase of Fusarium-Solani. Biochemical Journal 1971, 121, (3), 353-&.
47. Andersen, N.; Johansen, K. S.; Michelsen, M.; Stenby, E. H.; Krogh, K. B. R. M.; Olsson, L., Hydrolysis of cellulose using mono-component enzymes shows synergy during hydrolysis of phosphoric acid swollen cellulose (PASC), but competition on Avicel. Enzyme and Microbial Technology 2008, 42, (4), 362-370.
48. Szijarto, N.; Siika-aho, M.; Tenkanen, M.; Alapuranen, M.; Vehmaanpera, J.; Reczeya, K.; Viikari, L., Hydrolysis of amorphous and crystalline cellulose by heterologously produced cellulases of Melanocarpus albomyces. Journal of Biotechnology 2008, 136, (3-4), 140-147.
49. Stone, J. E.; Scallan, A. M.; Donefer, E.; Ahlgren, E., Digestibility as a Simple Function of a Molecule of Similar Size to a Cellulase Enzyme. Advances in Chemistry Series 1969, (95), 219-&.
50. Zhang, Y. H. P.; Cui, J. B.; Lynd, L. R.; Kuang, L. R., A transition from cellulose swelling to cellulose dissolution by o-phosphoric acid: Evidence from enzymatic hydrolysis and supramolecular structure. Biomacromolecules 2006, 7, (2), 644-648.
51. Jeoh, T.; Ishizawa, C. I.; Davis, M. F.; Himmel, M. E.; Adney, W. S.; Johnson, D. K., Cellulase digestibility of pretreated biomass is limited by cellulose accessibility. Biotechnology and Bioengineering 2007, 98, (1), 112-122.
52. Zhang, Y. H. P.; Lynd, L. R., Determination of the number-average degree of polymerization of cellodextrins and cellulose with application to enzymatic hydrolysis. Biomacromolecules 2005, 6, (3), 1510-1515.
53. Weimer, P. J.; Lopezguisa, J. M.; French, A. D., Effect of Cellulose Fine-Structure on Kinetics of Its Digestion by Mixed Ruminal Microorganisms Invitro. Applied and Environmental Microbiology 1990, 56, (8), 2421-2429.
54. Israilides, C. J.; Grant, G. A.; Han, Y. W., Sugar Level, Fermentability, and Acceptability of Straw Treated with Different Acids. Applied and Environmental Microbiology 1978, 36, (1), 43-46.
55. Um, B. H.; Karim, M. N.; Henk, L. L., Effect of sulfuric and phosphoric acid pretreatments on enzymatic hydrolysis of corn stover. Applied Biochemistry and Biotechnology 2003, 105, 115-125.
56. Moxley, G.; Zhu, Z. G.; Zhang, Y. H. P., Efficient sugar release by the cellulose solvent-based lignocellulose fractionation technology and enzymatic cellulose hydrolysis. Journal of Agricultural and Food Chemistry 2008, 56, (17), 7885-7890.
57. Thygesen, A.; Oddershede, J.; Lilholt, H.; Thomsen, A. B.; Stahl, K., On the determination of crystallinity and cellulose content in plant fibres. Cellulose 2005, 12, (6), 563-576.
58. De Souza, I. J.; Bouchard, J.; Methot, M.; Berry, R.; Argyropoulos, D. S., Carbohydrates in oxygen delignification. Part I: Changes in cellulose crystallinity. Journal of Pulp and Paper Science 2002, 28, (5), 167-170.
59. Gama, F. M.; Teixeira, J. A.; Mota, M., Cellulose Morphology and Enzymatic Reactivity—a Modified Solute Exclusion Technique. Biotechnology and Bioengineering 1994, 43, (5), 381-387.
60. Ardizzone, S.; Dioguardi, F. S.; Mussini, T.; Mussini, P. R.; Rondinini, S.; Vercelli, B.; Vertova, A., Microcrystalline cellulose powders: structure, surface features and water sorption capability. Cellulose 1999, 6, (1), 57-69.
61. Park, S.; Johnson, D. K.; Ishizawa, C. I.; Parilla, P. A.; Davis, M. F., Measuring the crystallinity index of cellulose by solid state C-13 nuclear magnetic resonance. Cellulose 2009, 16, (4), 641-647.
62. Bertran, M. S.; Dale, B. E., Determination of Cellulose Accessibility by Differential Scanning calorimetry. Journal of Applied Polymer Science 1986, 32, (3), 4241-4253.
63. Kimura, M.; Hatakeya. T; Nakano, J., Dsc Study on Recrystallization of Amorphous Cellulose with Water. Journal of Applied Polymer Science 1974, 18, (10), 3069-3076.
64. Hong, J.; Ye, X. H.; Zhang, Y. H. P., Quantitative determination of cellulose accessibility to cellulase based on adsorption of a nonhydrolytic fusion protein containing CBM and GFP with its applications. Langmuir 2007, 23, (25), 12535-12540.
65. Ramos, L. P.; Zandona, A.; Deschamps, F. C.; Saddler, J. N., The effect of Trichoderma cellulases on the fine structure of a bleached softwood kraft pulp. Enzyme and Microbial Technology 1999, 24, (7), 371-380.
66. Chen, Y.; Stipanovic, A. J.; Winter, W. T.; Wilson, D. B.; Kim, Y. J., Effect of digestion by pure cellulases on crystallinity and average chain length for bacterial and microcrystalline celluloses. Cellulose 2007, 14, (4), 283-293.
67. Lee, S. B.; Kim, I. H.; Ryu, D. D. Y.; Taguchi, H., Structural-Properties of Cellulose and Cellulase Reaction-Mechanism. Biotechnology and Bioengineering 1983, 25, (1), 33-51.
68. Ooshima, H.; Sakata, M.; Harano, Y., Adsorption of Cellulase from Trichoderma-Viride on Cellulose. Biotechnology and Bioengineering 1983, 25, (12), 3103-3114.
69. Väljamäe, P.; Sild, V.; Pettersson, G.; Johansson, G., The initial kinetics of hydrolysis by cellobiohydrolases I and II is consistent with a cellulose surface—erosion model. European Journal of Biochemistry 1998, 253, (2), 469-475.

70. Igarashi, K.; Wada, M.; Hori, R.; Samejima, M., Surface density of cellobiohydrolase on crystalline celluloses—A critical parameter to evaluate enzymatic kinetics at a solid-liquid interface. Febs Journal 2006, 273, (13), 2869-2878.
71. Segal, L.; Creely, J. J.; Martin, A. E.; Conrad, C. M., An empirical method for estimating the degree of crystallinity of native cellulose using the x-ray diffractometer. Textile Research Journal 1959, 29, 786-794.
72. Krassig, H. A., Cellulose: Structure, accessibility and reactivity. 1993, 11, Polymer Monographs, Gordon and Breach Science publisher, New York
73. Soltys, J.; Lisowski, Z.; Knapczyk, J., X-Ray Diffraction Study of the Crystallinity Index and the Structure of the Microcrystalline Cellulose. Acta Pharmaceutica Technologica 1984, 30, 174-180.
74. Bansal, P.; Hall, M.; Realff, M. J.; Lee, J. H.; Bommarius, A. B., Multivariate statistical analysis of X-ray data from cellulose: A new method to determine degree of crystallinity and predict hydrolysis rates. 101 Bioresource Technol. 4461-4471 (2010).
75. Hall et al., “Cellulose Crystallinity—a key predictor of the enzymatic hydrolysis rate”, FEBS Journal 277 (2010) 1571-1582.
Alzate, C. A. C., Toro, O. J. S. 2006. Energy consumption analysis of integrated flowsheets for production of fuel ethanol from lignocellulosic biomass. Energy, 31(13), 2447-2459.
Andersson, S., Serimaa, R., Paakkari, T., Saranpaa, P., Pesonen, E. 2003. Crystallinity of wood and the size of cellulose crystallites in Norway spruce (Picea abies). Journal of Wood Science, 49(6), 531-537.
Bansal, P., Hall, M., Realff, M. J., Lee, J. H., Bommarius, A. S. 2010. Multivariate statistical analysis of X-ray data from cellulose: a new method to determine degree of crystallinity and predict hydrolysis rates. Bioresour Technol, 101(12), 4461-71.
Boussaid, A., Robinson, J., Cai, Y., Gregg, D. J., Saddler, J. N. 1999. Fermentability of the hemicellulose-derived sugars from steam-exploded softwood (douglas fir). Biotechnol Bioeng, 64(3), 284-9.
Brown, R. E., Jarvis, K. L., Hyland, K. J. 1989. Protein measurement using bicinchoninic acid: elimination of interfering substances. Anal Biochem, 180(1), 136-9.
Bura, R., Bothast, R. J., Mansfield, S. D., Saddler, J. N. 2003. Optimization of SO₂-catalyzed steam pretreatment of corn fiber for ethanol production. Appl Biochem Biotechnol, 105-108, 319-35.
C. Cara, E. R., M. Ballesteros, P. Manzanares, J. Negro, E. Castro. 2008. Production of fuel ethanol from steam-explosion pretreated olive tree pruning. Fuel, 87(6), 692-700.
Cara, C., Ruiz, E., Ballesteros, I., Negro, M. J., Castro, E. 2006. Enhanced enzymatic hydrolysis of olive tree wood by steam explosion and alkaline peroxide delignification. Process Biochemistry, 41(2), 423-429.
Carrasco, C., Baudel, H., Penarrieta, M., Solano, C., Tejeda, L., Roslander, C., Galbe, M., Liden, G. 2011. Steam pretreatment and fermentation of the straw material “Paja Brava” using simultaneous saccharification and co-fermentation. J Biosci Bioeng, 111(2), 167-74.
Cherian, B. M., Leao, A. L., de Souza, S. F., Thomas, S., Pothan, L. A., Kottaisamy, M. 2010. Isolation of nanocellulose from pineapple leaf fibres by steam explosion. Carbohydrate Polymers, 81(3), 720-725.
Den Haan, R., Rose, S. H., Lynd, L. R., van Zyl, W. H. 2007. Hydrolysis and fermentation of amorphous cellulose by recombinant Saccharomyces cerevisiae. Metab Eng, 9(1), 87-94.
Dijkerman, R., Vervuren, M. B., Op Den Camp, H. J., van der Drift, C. 1996. Adsorption characteristics of cellulolytic enzymes from the anaerobic fungus Piromyces sp. strain E2 on microcrystalline cellulose. Appl Environ Microbiol, 62(1), 20-5.
Fan, L. T., Lee, Y. H., Beardmore, D. H. 1980. Mechanism of the Enzymatic-Hydrolysis of Cellulose—Effects of Major Structural Features of Cellulose on Enzymatic-Hydrolysis. Biotechnology and Bioengineering, 22(1), 177-199.
Galbe, M., Zacchi, G. 2007. Pretreatment of lignocellulosic materials for efficient bioethanol production. Adv Biochem Eng Biotechnol, 108, 41-65.
Gellerstedt, G. 2008. The Wood-Based Biorefinery: A New Concept for Green Chemicals and Materials. Proceedings of International Conference on Pulping, Papermaking and Biotechnology 2008: Icppb '08, Vol I, 9-15.
Hall, M., Bansal, P., Lee, J. H., Realff, M. J., Bommarius, A. S. 2010. Cellulose crystallinity—a key predictor of the enzymatic hydrolysis rate. FEBS J, 277(6), 1571-82.
Kumar, P., Barrett, D. M., Delwiche, M. J., Stroeve, P. 2009. Methods for Pretreatment of Lignocellulosic Biomass for Efficient Hydrolysis and Biofuel Production. Industrial & Engineering Chemistry Research, 48(8), 3713-3729.
Lu, Y., Yang, B., Gregg, D., Saddler, J. N., Mansfield, S. D. 2002. Cellulase adsorption and an evaluation of enzyme recycle during hydrolysis of steam-exploded softwood residues. Appl Biochem Biotechnol, 98-100, 641-54.
M. Ibrahim, F. A., W. El-Zawawy. 2010. Isolation and characterization of cellulose and lignin from steam-exploded lignocellulosic biomass. Bioresources, 5(1), 397-418.
Mosier, N., Wyman, C., Dale, B., Elander, R., Lee, Y. Y., Holtzapple, M., Ladisch, M. 2005. Features of promising technologies for pretreatment of lignocellulosic biomass. Bioresour Technol, 96(6), 673-86.
Moxley, G., Zhu, Z., Zhang, Y. H. 2008. Efficient sugar release by the cellulose solvent-based lignocellulose fractionation technology and enzymatic cellulose hydrolysis. J Agric Food Chem, 56(17), 7885-90.
Naik, S. N., Goud, V. V., Rout, P. K., Dalai, A. K. 2010. Production of first and second generation biofuels: A comprehensive review. Renewable & Sustainable Energy Reviews, 14(2), 578-597.
Nakagame, S., Chandra, R. P., Kadla, J. F., Saddler, J. N. 2011. The isolation, characterization and effect of lignin isolated from steam pretreated Douglas-fir on the enzymatic hydrolysis of cellulose. Bioresour Technol, 102(6), 4507-17.
Nguyen, Q. A., Tucker, M. P., Keller, F. A., Eddy, F. P. 2000. Two-stage dilute-acid pretreatment of softwoods. Appl Biochem Biotechnol, 84-86, 561-76.
Overend, R. P., Chornet, E. 1987. Fractionation of Lignocellulosics by Steam-Aqueous Pretreatments. Philosophical Transactions of the Royal Society of London Series a-Mathematical Physical and Engineering Sciences, 321(1561), 523-536.
P. Bansal, B. J. V., M. Hall, M. J. Realff, J. H. Lee, A. S. Bommarius. Elucidation of Cellulose Accessibility, Hydrolysability and Reactivity as the Major Limitations in the Enzymatic Hydrolysis of Cellulose. Bioresour Technol, accepted.
Pan, X., Xie, D., Gilkes, N., Gregg, D. J., Saddler, J. N. 2005. Strategies to enhance the enzymatic hydrolysis of pretreated softwood with high residual lignin content. Appl Biochem Biotechnol, 121-124, 1069-79.
Park, S., Baker, J. O., Himmel, M. E., Parilla, P. A., Johnson, D. K. 2010. Cellulose crystallinity index: measurement techniques and their impact on interpreting cellulase performance. Biotechnol Biofuels, 3, 10.
R. Sierra, A. S., C. Granda, M. Holtzapple. 2008. Producing fuels and chemicals from lignocellulosic biomass. SBE special section: biofuels.
Ramos, L. 2003. The chemistry involved in the steam treatment of lignocellulosic materials. Quim. Nova, 26(6), 863-871.
Segal L, C. J., Martin A, Conrad C. 1962. An empirical method for estimating the degree of crystallinity of native cellulose using the x-ray diffractometer. Textile Research Journal, 29, 786-794.
Soderstrom, J., Galbe, M., Zacchi, G. 2004. Effect of washing on yield in one- and two-step steam pretreatment of softwood for production of ethanol. Biotechnol Prog, 20(3), 744-9.
Sudo, K., Shimizu, K., Ishii, T., Fujii, T., Nagasawa, S. 1986. Enzymatic-Hydrolysis of Woods 0.9. Catalyzed Steam Explosion of Softwood. Holzforschung, 40(6), 339-345.
T. Yamashiki, T. M., M. Saitoh, K. Okajima, K. Kamide, T. Sawada. 1990. Characterisation of cellulose treated by the steam explosion method. Part 1: Influence of cellulose resources on changes in morphology, degree of polymerisation, solubility and solid structure. British Polymer Journal, 22(1), 73-83.
Taherzadeh, M. J., Karimi, K. 2008. Pretreatment of lignocellulosic wastes to improve ethanol and biogas production: a review. Int J Mol Sci, 9(9), 1621-51.
Tsao, G. 1999. Recent progress in bioconversion of lignocelluosics. Springer. Tu, M., Pan, X., Saddler, J. N. 2009. Adsorption of cellulase on cellulolytic enzyme lignin from lodgepole pine. J Agric Food Chem, 57(17), 7771-8.
Wang, K., Jiang, J. X., Xu, F., Sun, R. C. 2009. Influence of steaming explosion time on the physic-chemical properties of cellulose from Lespedeza stalks (Lespedeza crytobotrya). Bioresour Technol, 100(21), 5288-94.
Yin, S. Z., Wang, S. Q., Rials, T. G., Kit, K. M., Hansen, M. G. 2007. Polypropylene composites filled with steam-exploded wood fibers from beetle-killed loblolly pine by compression-molding. Wood and Fiber Science, 39(1), 95-108.
Zhao, Y., Wang, Y., Zhu, J. Y., Ragauskas, A., Deng, Y. 2008. Enhanced enzymatic hydrolysis of spruce by alkaline pretreatment at low temperature. Biotechnol Bioeng, 99(6), 1320-8.
Zhu, J. Y., Pan, X., Zalesny, R. S., Jr. 2010. Pretreatment of woody biomass for biofuel production: energy efficiency, technologies, and recalcitrance. Appl Microbiol Biotechnol, 87(3), 847-57.
Zhu, J. Y., Pan, X. J. 2010. Woody biomass pretreatment for cellulosic ethanol production: Technology and energy consumption evaluation. Bioresour Technol, 101(13), 4992-5002.

What is claimed is:

1. A method for producing glucose for fermentation said method comprising:
(a) treating a biomass comprising a lignocellulosic material with a mixture comprising SO2 and steam at reaction conditions sufficient to produce a composition mixture comprising cellulose suitable for enzymatic hydrolysis;
(b) enzymatically hydrolyzing at least a portion of the cellulose of step (a) under conditions sufficient to form a composition comprising glucose; and
(c) utilizing glucose in an aerobic or anaerobic fermentation;
wherein the temperature, residence time, and SO2 concentration of step (a) are selected by calculating a crystallinity index (CrI) of said biomass and then using the calculated crystallinity index as an indicator of enzymatic hydrolysis rate.

2. The method of claim 1 which further comprises treating the biomass comprising a lignocellulosic material with an aqueous acid prior to enzymatic hydrolysis.

3. The method of claim 2 wherein the aqueous acid is phosphoric acid.

4. The method of claim 1 wherein the temperature, residence time, and SO2 concentration employed in step (a) are selected to reduce the CrIcel (%) of the treated biomass by at least about 5% from that of untreated biomass according to
$${CrI}_{cel} = \frac{CrI}{{F}_{c}}$$
where CrI_{cel} is the CrI of cellulose fraction in treated biomass, F_{c} is the percentage content of cellulose in the sample, and CrI is crystallinity index.

5. The method of claim 1 wherein the temperature, residence time, and SO2 concentration employed in step (a) are selected to reduce the CrIcel (%) of the treated biomass by at least about 12% from that of untreated biomass according to
$${CrI}_{cel} = \frac{CrI}{{F}_{c}}$$
where CrI_{cel} is the CrI of cellulose fraction in treated biomass, F_{c} is the percentage content of cellulose in the sample, and CrI is crystallinity index.

6. The method of claim 1 wherein the temperature, residence time, and SO2 concentration employed in step (a) are selected to reduce the CrIcel (%) of the treated biomass by at least about 18% from that of untreated biomass according to
$${CrI}_{cel} = \frac{CrI}{{F}_{c}}$$
where CrI_{cel} is the CrI of cellulose fraction in treated biomass, F_{c} is the percentage content of cellulose in the sample, and CrI is crystallinity index.

7. The method of claim 1 wherein the temperature, residence time, and SO2 concentration employed in step (a) are selected to yield a severity index (R) of from about 2.5 to about 4.0 wherein R is calculated according to the following Equation 1:
$$R = {log}_{10} ( t {e}^{\frac{T - 100}{14.75}} ) - pH \qquad ( 1 )$$
where t is residence time (min) and T is temperature (° C.).

8. The method of claim 1 wherein the temperature, residence time, and SO2 concentration employed in step (a) are selected to yield a severity index (R) of from about 2.8 to about 3.8 wherein R is calculated according to the following Equation 1:
$$R = {log}_{10} ( t {e}^{\frac{T - 100}{14.75}} ) - pH \qquad ( 1 )$$
where t is residence time (min) and T is temperature (° C.).

9. The method of claim 1 wherein the temperature, residence time, and SO2 concentration employed in step (a) are selected to yield a severity index (R) of from about 3.1 to about 3.6 wherein R is calculated according to the following Equation 1:
$$R = {log}_{10} ( t {e}^{\frac{T - 100}{14.75}} ) - pH \qquad ( 1 )$$
where t is residence time (min) and T is temperature (° C.).

10. The method of claim 1 wherein the temperature, residence time, and SO2 concentration employed in step (a) are selected to yield a severity index (R) of from about 3.2 to about 3.5 wherein R is calculated according to the following Equation 1:
$$R = {log}_{10} ( t {e}^{\frac{T - 100}{14.75 }} ) - pH \qquad ( 1 )$$
where t is residence time (min) and T is temperature (° C.).

11. The method of claim 1 wherein the temperature, residence time, and SO2 concentration employed in step (a) are selected to yield a severity index (R) of at least about 3.2 and wherein an acid insoluble lignin (AIL) content of the treated biomass is less than about 60%.

12. The method of claim 1 wherein the glucose is fermented to a product selected from the group consisting of alcohols, fatty alcohols, hydrocarbons, fatty acids, tryglycerides, terpenes, and combinations thereof.

13. The method of claim 1 wherein the lignocellulosic material is selected from the group consisting of switchgrass, loblolly pine, bagasse, corn stover, poplar tree, miscanthus, and mixtures thereof.

14. The method of claim 1 wherein the lignocellulosic material is selected from the group consisting of loblolly pine, poplar tree, spruce, eucalyptus, and mixtures thereof and wherein the temperature, residence time, and SO2 concentration employed in step (a) are selected such that acid insoluble lignin (AIL) content of the treated biomass is between about 25 and about 70%.

15. The method of claim 1 wherein the lignocellulosic material is selected from the group consisting of loblolly pine, poplar tree, spruce, eucalyptus, and mixtures thereof and wherein the temperature, residence time, and SO2 concentration employed in step (a) are selected such that acid insoluble lignin (AIL) content of the treated biomass is less than about 40%.

16. The method of claim 1 wherein the lignocellulosic material is loblolly pine.

17. The product of the method of claim 1.

18. A method for producing glucose for fermentation, said method comprising:
(a) treating a lignocellulosic material at acidic reaction conditions sufficient to produce a composition mixture comprising cellulose suitable for enzymatic hydrolysis; and
(b) enzymatically hydrolyzing at least a portion of the cellulose of step (a) under conditions sufficient to form a composition comprising glucose;
wherein the acidic reaction conditions employed in step (a) are selected to reduce the CrIcel (%) of the treated biomass by at least about 6% from that of untreated biomass according to
$${CrI}_{cel} = \frac{CrI}{{F}_{c}}$$
where CrI_{cel} is the CrI of cellulose fraction in treated biomass, F_{c} is the percentage content of cellulose in the sample, and CrI is crystallinity index and wherein the acid insoluble lignin (AIL) content of the treated biomass is less than about 60%.

19. The method of claim 18 wherein the lignocellulosic material comprises loblolly pine and wherein the acid reaction conditions employed in step (a) are selected to yield a severity index (R) of at least about 3.2 wherein R is calculated according to the following Equation 1:
$$R = {log}_{10} ( t {e}^{\frac{T - 100}{14.75}} ) - pH \qquad ( 1 )$$
where t is residence time (min) and T is temperature (° C.).

20. The method of claim 19 wherein the lignocellulosic material is treated with a mixture comprising SO2 and steam in step (a).
